(19) United States
(12) Patent Application Publication
(10) Pub. No.: US 2025/0154570 A1
(43) Pub. Date: May 15, 2025

(54) MATERIALS AND METHODS FOR LARGE-SCALE SPATIAL TRANSCRIPTOMICS

(71) Applicant: The University of Chicago, Chicago, IL (US)

(72) Inventor: NICOLAS CHEVRIER, Chicago, IL (US)

(21) Appl. No.: 18/837,656

(22) PCT Filed: Feb. 14, 2023
(86) PCT No.: PCT/US23/13010

§ 371 Date: Aug. 12, 2024

Related U.S. Application Data
(60) Provisional application No. 63/309,795, filed on Feb. 14, 2022.

(51) Int. Cl. C12Q 1/6841; C12Q 1/6837

(52) U.S. Cl. CPC C12Q 1/6841; C12Q 1/6837

(57) ABSTRACT

The present disclosure relates to materials and methods for large-scale spatial transcriptomics. In particular, the disclosure provides methods for producing systems for spatial transcriptomics, along with materials and methods for determining the spatial location of a desired nucleic acid, such as RNA, within a tissue sample.

### STATEMENT REGARDING RELATED APPLICATIONS

This application claims priority to U.S. Provisional Patent Application No. 63/309,795, filed Feb. 14, 2022, the entire contents of which are incorporated herein by reference for all purposes.

### FIELD

The present disclosure relates to materials and methods for large-scale spatial transcriptomics. In particular, the disclosure provides materials and methods for determining the spatial location of a desired nucleic acid, such as RNA, within a tissue sample.

### BACKGROUND

Currently used methods for determining the spatial location of gene expression in a tissue sample are limited by various factors, including poor resolution and limited ability to use large tissue samples. Accordingly, improved methods for determining the spatial location of nucleic acids in a tissue sample are needed, in particular methods that permit detection of the spatial location of a nucleic acid with high resolution and methods that allow for large tissue samples to be utilized.

### SUMMARY

In some aspects, provided herein are methods of producing systems for spatial detection of nucleic acid in a tissue sample. In some embodiments, methods for producing a system for spatial detection of nucleic acid in a tissue sample comprise providing a support comprising an array of surface probes and performing reactions directly on the support, thereby creating a system comprising spots of capture probes. In some embodiments, each surface probe on the support comprises a first anchor sequence, a spatial barcode, and a second anchor sequence. In some embodiments, the method comprises hybridizing a first complementary oligonucleotide to the first anchor sequence of each surface probe and hybridizing a second complementary oligonucleotide to the second anchor sequence of each surface probe. In some embodiments, the second oligonucleotide comprises a nucleic acid capture region (e.g. a capture domain) and a unique molecular identifier. In some embodiments, the method comprises performing an extension-ligation reaction on the support. The extension-ligation reaction performed directly on the support is also referred to herein as an “on-slide” reaction. In some embodiments, the extension-ligation reaction comprises extending the first complementary nucleotide with a sequence complementary to the spatial barcode of the surface probe, and ligating the extended first complementary nucleotide and the second complementary nucleotide together to form a contiguous capture oligonucleotide. The contiguous capture oligonucleotide comprises a nucleic acid capture region, a unique molecular identifier, a first anchor sequence, a spatial barcode, and a second anchor sequence.

In some embodiments, for each surface probe, the first anchor sequence, the second anchor sequence, and the spatial barcode each comprise 10-30 nucleotides. In some embodiments, for each surface probe the first anchor sequence comprises 20-30 nucleotides, the second anchor sequence comprises 10-20 nucleotides, and the spatial barcode comprises 15-25 nucleotides. In some embodiments, for each surface probe the first anchor sequence comprises 24 nucleotides, the second anchor sequence comprises 16 nucleotides, and the spatial barcode comprises 18 nucleotides. In some embodiments, for each surface probe the second anchor sequence comprises 40% to 60% guanosine and/or cytosine (G/C) bases. In some embodiments, the nucleic acid capture region comprises at least 10 deoxythymidine residues.

In some embodiments, performing the extension ligation reaction on the support comprises adding a DNA polymerase and a DNA ligase to the support under conditions such that the reverse complement of the spatial barcode sequence is synthesized and ligated to the first complementary nucleotide and to the second complementary nucleotide, thereby forming the continuous capture probe.

In some aspects, provided herein are systems for spatial detection of nucleic acid. In some embodiments, provided herein are systems for spatial detection of nucleic acid in a tissue sample. For example, in some embodiments provided herein are systems for spatial detection of RNA in a tissue sample.

In some embodiments, the system comprises a plurality of spots immobilized on a support. In some embodiments, each spot comprises a plurality of capture oligonucleotides. In some embodiments, each capture oligonucleotide comprises a nucleic acid capture region, a unique molecular identifier, a first anchor sequence, and a spatial barcode. In some embodiments, each capture oligonucleotide in a single spot comprises the same spatial barcode. In some embodiments, the spatial barcode for each distinct spot is unique. In some embodiments, each capture oligonucleotide further comprises a second anchor sequence.

In some embodiments, the support comprises a working surface area of at least 2 cm². Accordingly, the systems and methods described herein facilitate spatial detection of nucleic acids in a large (e.g. greater than 2 cm² area) tissue slice. In some embodiments, the support comprises a working surface area of at least 5 cm². In some embodiments, the support comprises a working surface area of at least 10 cm². In some embodiments, the working surface area is substantially circular in shape. In some embodiments, the working surface area is substantially rectangular in shape. In some embodiments, the working surface area is substantially square shaped.

In some embodiments, the working surface area of the support comprises at least 200 spots/mm². In some embodiments, the working surface area of the support comprises at least 400 spots/mm². In some embodiments, the working surface area of the support comprises at least 800 spots/mm². In some embodiments, the nucleic acid capture region comprises at least 10 deoxythymidine residues.

In some aspects, provided herein are methods of making a system for spatial detection of nucleic acid in a tissue sample. In some embodiments, the method comprises providing a support comprising an array of surface probes, each surface probe comprising a first anchor sequence, a spatial barcode, and a second anchor sequence. In some embodiments, the method further comprises hybridizing a first complementary oligonucleotide to the first anchor sequence of each surface probe. In some embodiments, the method further comprises hybridizing a second complementary oligonucleotide to the second anchor sequence of each surface probe. In some embodiments, the second oligonucleotide further comprises a nucleic acid capture region and a unique molecular identifier. In some embodiments, the method further comprises extending the first complementary nucleotide with a sequence complementary to the spatial barcode of the surface probe, such that the first complementary nucleotide and the second complementary nucleotide form a contiguous capture oligonucleotide. The capture oligonucleotide comprises a nucleic acid capture region, a unique molecular identifier, a first anchor sequence, a spatial barcode, and a second anchor sequence.

In some aspects, provided herein are kits. In some embodiments, provided herein is a kit comprising a system as described herein.

In some embodiments, provided herein are methods for spatial detection of nucleic acid in a tissue sample. In embodiments, provided herein are methods for spatial detection of RNA in a tissue sample. In some embodiments, the method comprises contacting a system as described herein with a tissue sample.

In some embodiments, the method for spatial detection of RNA in a tissue sample, comprising contacting a system described herein with a tissue sample, such that RNA within the tissue sample to binds to the capture oligonucleotides. In some embodiments, the method further comprises reverse-transcribing the bound RNA to generate cDNA. In some embodiments, the method further comprises sequencing the cDNA. In some embodiments, the method further comprises correlating the spatial barcode for each sequenced cDNA molecule with the location of the spot on the support having a corresponding spatial barcode. In some embodiments, the method further comprises imaging the tissue and/or staining the tissue before or after sequencing the nucleic acid molecules. In some embodiments, the method further comprises determining the spatial location of the sequenced cDNA molecules within the tissue sample by correlating the location of the spot on the support with a corresponding location within the tissue sample.

In some embodiments, provided herein is a method for spatial detection of RNA in a tissue sample performed using a hybridized tissue sample. In some embodiments, the method comprises hybridizing a first probe and a second probe to a target RNA sequence in a tissue sample, wherein the first probe and the second probe each comprise an RNA binding region complementary to the target RNA sequence. In some embodiments, the method further comprises ligating the RNA binding region of the first probe to the RNA binding region of the second probe, thereby forming an extended probe that is hybridized to the target RNA sequence in the tissue sample. In some embodiments, the method comprises contacting the tissue sample with a system described herein, such that extended probes bind to the capture oligonucleotides, reverse-transcribing the bound extended probes to generate cDNA, and sequencing the cDNA. In some embodiments, the first probe further comprises a capture oligonucleotide binding region complementary to the nucleic acid capture domain of a capture oligonucleotide. In some embodiments, the second probe further comprises a sequencing handle. In some embodiments, the tissue sample is a fresh frozen sample or a formalin-fixed, paraffin-embedded (FFPE) tissue sample. In some embodiments, the tissue sample is an FFPE tissue sample, and the method further comprises deparaffinizing the tissue sample and decrosslinking RNA in the tissue sample prior to hybridizing the first probe and the second probe to the target RNA sequence.

In some embodiments, the method further comprises correlating a spatial barcode for each sequenced cDNA molecule with the location of the spot on the support having a corresponding spatial barcode. In some embodiments, the method further comprises imaging the tissue and/or staining the tissue before or after sequencing the nucleic acid molecules. In some embodiments, the method comprises imaging and/or staining the tissue before hybridizing the first and second probe to the target RNA sequence in the tissue. In some embodiments, the method comprises imaging and/or staining the tissue after hybridizing the first and second probe to the target RNA sequence in the tissue. In some embodiments, the method further comprises determining the spatial location of the sequenced cDNA molecules within the tissue sample by correlating the location of the spot on the support with a corresponding location within the tissue sample. In some embodiments, the tissue sample has a surface area of at least 2 cm². In some embodiments, the tissue sample has a surface area of at least 5 cm². In some embodiments, the tissue has a surface area of at least 10 cm².

In some embodiments, provided herein is a method for spatial detection of RNA in a tissue sample, comprising obtaining a tissue sample having a surface area of at least 2 cm², wherein the tissue sample is stabilized on an adhesive film, and transferring the tissue sample from the adhesive film to a system described herein. In some embodiments, the method comprises transferring the tissue sample from the adhesive film to a system described herein, such that RNA within the tissue sample to binds to the capture oligonucleotides. In some embodiments, the method further comprises reverse-transcribing the bound RNA to generate cDNA, and sequencing the cDNA. In some embodiments, transferring the tissue sample comprises mounting the stabilized tissue sample on the support and dissolving the adhesive film in hexane, thereby transferring the tissue sample from the adhesive film to the support. In some embodiments, the method further comprises correlating a spatial barcode for each sequenced cDNA molecule with the location of the spot on the support having a corresponding spatial barcode. In some embodiments, the method further comprises imaging the tissue and/or staining the tissue after transferring the tissue sample from the adhesive film to the support. In some embodiments, the tissue is imaged and/or stained before or after sequencing the cDNA. In some embodiments, the method further comprises determining the spatial location of the sequenced cDNA molecules within the tissue sample by correlating the location of the spot on the support with a corresponding location within the tissue sample. In some embodiments, the tissue sample has a surface area of at least 2 cm². In some embodiments, the tissue has a surface area of at least 5 cm². In some embodiments, the tissue has a surface area of at least 10 cm².

### DESCRIPTION OF THE DRAWINGS

FIG. 1 shows an exemplary method for preparing and using the systems for spatial detection of nucleic acid described herein.

FIG. 2 shows an exemplary embodiment of using the systems and methods described herein using a sagittal slice from an entire organism (in this figure, a mouse).

FIG. 3 shows slices from a whole mouse. Slices shown are the unstained tissue contained within the block face, a 10 μm section, a 10 μm section after H&E staining, and a 10 μm section showing the number of unique molecular identifiers per spot.

FIG. 4 shows Uniform Manifold Approximation and Projection (UMAP) plots visualizing gene expression in a whole mouse slice. Clusters of gene expression are shown in the “UMAP Clusters” slice.

FIG. 5 shows gene expression-based classification of organs using whole-tissue RNA-sequencing.

FIG. 6 shows individually stained organ clusters from muscle, brain, subdermal fascia, and skin.

FIG. 7 shows individually stained organ clusters from fat, liver, and skin.

FIG. 8 shows individually stained clusters from lung, kidney, brain, and lymphoid.

FIG. 9 shows individually stained clusters from large blood vessels, heart, kidney, and colon.

FIG. 10 shows individually stained clusters from stomach and brain.

FIG. 11 shows individually stained clusters 25, 26, and 27.

FIG. 12A shows an exemplary embodiment of a method described herein wherein a cryomacrotome is modified to be suited for obtaining intact slices from a whole adult mouse. The slicing method employs an adhesive film to stabilize the slice and transfer the slice to a support. In this embodiment, the blade holder and blade on a Leica CM3600 XP cryomacrotome is modified and used to slice an embedded whole adult mouse (block face). Image 1 shows the block face and block holder. Image 2 shows the adhesive film held by forceps above the block. Image 3 shows the application of the film. Images 4, 5, and 6 show sectioning while retrieving a 10 micron whole-mouse section from the film. FIG. 12B shows H&E images from serial sagittal sections from an adult mouse. FIG. 12C-12D show annotated H&E images. Scale bars in FIG. 12 D are 250 microns for insets a-c and 500 microns for inset d.

FIGS. 13A-13D show that transfer of histological sections onto a glass slide preserves RNA and allows for 3D analysis. FIG. 13A shows an overview of an exemplary transfer procedure using hexane to dissolve the adhesive film (step 2). FIG. 13B shows total RNA quality before (left) and after (right) transfer of mouse thymus sections (10 microns). FIG. 13C shows an example of serial sectioning using mouse B16 tumors (approximately 100 mm³) and FIG. 13D shows an example of serial sectioning using lymph node. 3 and 5 sections, for tumor and lymph node samples, respectively, were skipped in between serial sections. 3D reconstructions are shown on the right side for each organ type.

FIGS. 14A-14C show custom microarrays for ST profiling. FIG. 14A shows an exemplary embodiment of a custom microarray as described herein. The microarray was customized and synthesized. In this example, the custom microarray was synthesized by Agilent. The exemplary customized array comprises 974,016 spots arranged in 1,068 rows and 912 columns across 11.31 cm² in surface area. FIG. 14B shows on-slide mRNA capture probe assembly by hybridization (step 1) of the indicated probes, followed by on-slide extension/ligation (gap-fill, step 2). FIG. 14C shows PAGE analysis of the oligo products obtained after the indicated on-slide assembly procedures (a through d). Anchor 1 (24-mer); Spatial Barcode (18-mer); Anchor 2 (16-mer); Anchor 2-UMI-dT (16+17+32=65-mer); Overextended Anchor 1 (24+18+16=58-mer); Extended Anchor 1 (24+18=42-mer); Capture probe (24+18+16+17+32=107-mer).

FIGS. 15A-H show ST data generated using a custom microarray (also referred to as “in-house” vs. the commercially available Visium array. FIG. 15A shows spot coverage (18.8% Visium, 60.1% in-house). FIG. 15B-15F show bar plots of indicated metrics from the array (FIG. 15B) and kidney ST data (FIG. 15C-F). FIG. 15G-15H show H&E images (left) and indicated metrics overlaid on the H&E image including UMIs and genes per spot and spatial clusters (colors) obtained from each ST dataset.

FIGS. 16A-16F show whole-mouse ST data generated using a custom microarray as described herein. FIG. 16A shows whole-mouse sectioning, H&E image, UMIs/spot, and clustering (left to right). Colors, organs; scale bar, 5 mm. FIG. 16B shows examples of organ-specific genes. FIG. 16C-16D show brain (FIG. 16C) and kidney (FIG. 16D) clustering an example genes. FIG. 16E-16F show nucleic per spot for indicated organs (FIG. 16F) calculated using DAPI stained whole mouse sections on a customized ST Array hybridized with Cy3-labeled probes (FIG. 16E, brain area).

FIG. 17 shows an overview of optimization methods for the sequence of custom array probes. Shown are a probe on the array glass (left) and 9 design variants that can be tested for Anchor 2. The spatial barcode sequence will vary according to the sequence of Anchor 2.

FIG. 18 shows a bar graph showing array ligation biases observed. Bar plots of the number of UMIs captured by spot across the custom ST array were grouped and normalized by the last base of the spatial barcode in arrays prepared with a 90 minute incubation (left) or an overnight incubation step (right) for the gap-fill reaction during on-slide capture probe assembly, as detailed in FIG. 14.

FIG. 19 shows mouse kidney permeabilization. Shown are H&E (left) and fluorescence (right) images of mouse kidney sections. Incubation times indicate permeabilization time in a pepsin solution using the Visium Tissue Optimization Kit.

FIG. 20 shows an exemplary method for ST in FFPE tissue. Following suitable deparaffinization and RNA decrosslinking steps, probe pairs hybridize to target mRNA in FFPE section (top). After ligation and RNA digestion (middle), ligated probe pairs are captured on custom ST arrays and extended. For example, ligated probe pairs can be captured via oligo-dT and extended 3′. Resulting DNA is processed for sequencing. A1=Anchor 1; SB=spatial barcode, A2=Anchor 2 (FIG. 14).

FIG. 21 shows an exemplary method for dual mRNA/protein ST. Shown in a spleen section immunolabeled with DNA-barcoded antibodies (Ab) and permeabilized to release mRNA (purple) for capture on the ST array (left). cDNA and Ab barcode tags are synthesized and processed for sequencing (middle) and downstream analysis (right). A1=Anchor 1; SB=spatial barcode, A2=Anchor 2 (FIG. 14).

FIG. 22 shows an exemplary method for dual mRNA/VDJ ST. After mRNA capture on the ST Array (left), cDNA is synthesized and amplified. In this exemplary method, cDNA is synthesized by reverse transcription (RT) and amplified by single-primer PCR (SPA). Resulting full-length cDNA is enriched for T cell receptor (TR) and B cell receptor (IG) cDNAs. In this example, cDNAS are enriched by pulling down biotinylated probes (middle). Suitable sequencing analysis can then be performed. In this example, sequencing is performed by long-read nanopore sequencing (right) am analysis. A1=Anchor 1; SB=spatial barcode, A2=Anchor 2 (FIG. 14).

FIG. 23 is a frequency bar plot showing TRBV gene usage. Indicated TRBV genes are on the X axis as measured using TCR-seq libraries adapted for long (Nanopore) or short (Illumina) sequencing reads.

FIG. 24 shows whole-mount human colon with tumor sectioning and H&E annotation. Shown is a partial colon resected from a colon cancer patient (˜50 cm in length). A full ring was cut (left panel: white dashed line), fresh-frozen, embedded, sectioned with custom adhesive film and H&E stained (second panel from left). Tissue subregions and cell types were annotated by a gastrointestinal pathologist (Lindsay Alpert, Letter of Support). Middle to right panels show zoomed in subregions as indicated by black boxes and dashed arrows. a, infiltrating lymphocytes; b, lymphovascular space; c, necrotic cells and debris; d, malignant gland; e, desmoplasmic stroma. Circles (right-most panel) indicate the following cell types: green=lymphocyte, yellow=eosinophil, red=tumor cell, brown=endothelial cell, orange=globlet cell, blue=stromal cell.

FIG. 25A-25B show that large format human colon sections yield high quality RNA. (A) Healthy human colon tissue was fresh-frozen and sectioned (left) using custom adhesive tape followed by H&E staining and imaging (right). (B) Tapestation trace of total RNA extracted from transferred human colon section from A.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “oligonucleotide” refers to a molecule comprising two or more nucleotides (e.g., deoxyribonucleotides or ribonucleotides). The terms “nucleotide” and “base” are used interchangeably when used in reference to a nucleic acid sequence, unless indicated otherwise herein. The term “nucleotide” or “base” is inclusive of naturally occurring and synthetic bases. “Oligonucleotides” are typically less than 200 residues long (e.g., between 2 and 100 nucleotides), however, as used herein, the term is also intended to encompass longer polynucleotide chains.

### DETAILED DESCRIPTION

The architecture of a tissue determines its function and malfunction in health and disease. Therefore, technologies to image and measure the cells and molecules organized in tissues are fundamental to biomedical research and clinical pathology. Recent advances in the field of spatially resolved transcriptomics (ST) have enabled the sequencing of the transcriptome associated with specific areas of tissue sections. ST technologies have not spread beyond their labs of origin due to, for example, the need for complex instrumentation and expertise and the lack of access to key reagents, instruments, or well-documented protocols. One exception is the commercially available kit (Visium, 10× Genomics) which has been adopted by several institutions, but this commercial platform remains limited by its low resolution (55-μm spots with 100 μm center-to-center distances between them), small surface area (<0.5 cm²), and high cost (˜$25/mm² without sequencing). In addition, the limitations of this commercial platform compound with the issues of standard freeze-sectioning techniques that are prone to section loss and damage. Taken together, these gaps in ST and histology technologies prevent spatiomolecular analyses across many samples, small or large, for detailed studies of temporal processes, patient cohorts, or spatial atlases in two or three dimensions. The systems for spatial detection of nucleic acid in a tissue sample and methods of making systems for spatial detection of nucleic acid in a tissue sample provided herein address these and other issues. In particular, the systems and methods described herein enable high resolution, high sensitivity, and affordable methods for spatial detection of nucleic acid in a tissue sample, including whole animal tissue samples.

The systems and methods of producing systems for spatial transcriptomics provided herein enable analysis of small-to-large samples ranging from a biopsy to whole-mount sections of human organs or adult rodents. Resulting data sets encompass large numbers of cells from a single experiment. For example, data sets can encompass ˜4-6M cells in a whole, adult mouse section or ˜1-2M cells in a whole mount human colon section. These large format arrays also enable high throughput ST profiling, enabling generation of 3D ST data using serial sectioning techniques, the creation of ST atlases across model organisms, and/or the cost-effective processing of fixed samples from large cohorts of patients. The systems and methods described herein use an adhesive film to support sections from tissues or larger samples such as whole-mount rodents or human organs, while preserving RNA for ST compatibility and dramatically increasing the quality and reproducibility of sectioning (little to no section loss).

In some aspects, provided herein are systems for spatial detection of nucleic acid in a tissue sample. In some aspects, provided herein are methods for producing systems for spatial detection of nucleic acid in a tissue sample. Spatial detection of nucleic acid in a tissue sample is also referred to herein as “spatial transcriptomics” or “ST”. The systems for spatial detection of nucleic acid in a tissue sample provided herein are also referred to herein as an “array”, a “custom array”, or an “ST array”. In some embodiments, the systems are used for spatial detection of RNA in a tissue sample. In some embodiments, the substrates may be used for spatial detection of RNA transcripts (e.g., mRNA) in a tissue sample.

The tissue sample can be any suitable tissue sample, including human tissues and tissues from non-human subjects (e.g. vertebrates).

In some embodiments, provided herein are systems for spatial detection of nucleic acids in a tissue sample. In some embodiments, the system comprises a plurality of spots immobilized on a support. In some embodiments, each spot comprises a plurality of capture oligonucleotides. In some embodiments, each capture oligonucleotide comprises a nucleic acid capture region, a unique molecular identifier, a first anchor sequence, and a spatial barcode. In some embodiments, each capture oligonucleotide further comprises a second anchor sequence. In some embodiments, the 5′ end of each capture oligonucleotide is proximal to the support, and the 3′ end of each capture oligonucleotide is furthest away from the support. In some embodiments, each capture oligonucleotide comprises, from 5′ end to 3′ end, a first anchor sequence, a spatial barcode, a second anchor sequence, a unique molecular identifier, and a nucleic acid capture region. In some embodiments, the capture oligonucleotides are immobilized to the support via hybridization with surface probes that are immobilized on the support.

In some embodiments, each capture oligonucleotide in a single spot comprises the same spatial barcode. In some embodiments, the spatial barcode for each distinct spot is unique. Accordingly, in some embodiments no two spots on the support comprise capture oligonucleotides comprising the same spatial barcode.

In some embodiments, the support comprises a working surface area of at least 1 cm². The term “working surface area” as used herein refers to a surface area of the support (e.g. a section of the support) that is substantially occupied by spots. In other words, the “working surface area” refers to the area of the support that is densely populated with spots (e.g. contains at least 200 spots/mm²). The “working surface area” of the support comprises a portion of the total surface area of the support. For example, the support may comprise a total surface area that is larger than the working surface area, thereby providing regions on the support that can be touched without impacting the capture probes immobilized within the “working surface area” of the support. In some embodiments, the support comprises a working surface area of at least 1 cm², 2 cm², 3 cm², 4 cm², 5 cm², 6 cm², 7 cm², 8 cm², 9 cm², 10 cm², 11 cm², 12 cm², 13 cm², 14 cm², 15 cm², 16 cm², 17 cm², 18 cm², 19 cm², or 20 cm². The support may comprise any suitable shape, including square, rectangular, hexagonal, or circular. The working surface area of the support may be any suitable shape, including substantially square, substantially rectangular, substantially circular, etc. In some embodiments, the working surface area is sufficient large to enable spatial transcriptomic analysis of an equally large tissue sample. For example, in some embodiments the working surface area is at least 2 cm² (e.g. at least 2 cm², at least 3 cm², at least 5 cm², at least 7 cm², at least 10 cm², at least 12 cm²) and the tissue sample has a surface area of approximately the same size as the working surface area. In some embodiments, the working surface area of the support enables spatial transcriptomic analysis of a tissue sample (e.g. a tissue slice) having a surface area of at least 2 cm² (e.g. at least 2 cm², at least 3 cm², at least 4 cm², at least 5 cm², at least 6 cm², at least 7 cm², at least 8 cm², at least 9 cm², at least 10 cm², at least 11 cm², at least 12 cm², at least 13 cm², at least 14 cm², at least 15 cm², at least 16 cm², at least 17 cm², at least 18 cm², at least 19 cm², or about 20 cm².

The support may comprise any suitable material, including glass and/or plastics. The material may be porous or non-porous. In some embodiments, the support comprises a material selected from glass, silicon, poly-L-lysine coated materials, nitrocellulose, polystyrene, cyclic olefin copolymers (COCs), cyclic olefin polymers (COPs), polypropylene, polyethylene and polycarbonate. In some embodiments, the support comprises a solid material, such as glass and/or plastic. In other embodiments, the support comprises a gel.

In some embodiments, the working surface area of the support comprises at least 200 spots/mm². In some embodiments, the working surface area of the support comprises at least 200 spots/mm², at least 250 spots/mm², at least 300 spots/mm², at least 350 spots/mm², at least 400 spots/mm², at least 450 spots/mm², at least 500 spots/mm², at least 550 spots/mm², at least 600 spots/mm², at least 650 spots/mm², at least 700 spots/mm², at least 750 spots/mm², or at least 800 spots/mm².

In some embodiments, the support comprises at least 10,000 total spots. For example, the support may comprise at least 10,000, at least 15,000, at least 20,000, at least 25,000, at least 30,000, at least 35,000, at least 40,000, at least 45,000, or at least 50,000 total spots.

In some embodiments, each spot comprises at least 500 capture oligonucleotides. For example, each spot may comprise at least 500, at least 600, at least 700, at least 800, at least 900, at least 1000, at least 1100, at least 1200, at least 1300, at least 1400, at least 1500, at least 1600, at least 1700, at least 1800, at least 1900, at least 2000, at least 2100, at least 2200, at least 2300, at least 2400, at least 2500, at least 2600, at least 2700, at least 2800, at least 2900, or at least 3000 capture oligonucleotides.

In some embodiments, each capture oligonucleotide comprises a nucleic acid capture domain. The terms “nucleic acid capture domain”, “capture domain”, “nucleic acid capture region”, and “capture region” are used interchangeably herein. In some embodiments, the nucleic acid capture region comprises a poly-T oligonucleotide (e.g. a series of consecutive deoxythymidine residues linked by phosphodiester bonds). A poly-T oligonucleotide may also be referred to as oligo (dT) or an oligo (dT) tail. For example, in some embodiments, the capture domain comprises a poly-T oligonucleotide comprising at least 10 deoxythymidine residues. In some embodiments, the nucleic acid capture region comprises nucleotides which are analogous to poly-T and retain the functional property of binding to the poly-A tail of mRNA. For example, the capture domain may comprise a poly-U oligonucleotide.

In some embodiments, each capture oligonucleotide comprises a unique molecular identifier (UMI). Each UMI comprises a nucleotide sequence. The UMI may be a nucleotide sequence of any suitable length. In some embodiments, the UMI comprises 5-50 nucleotides. In some embodiments, the UMI comprises 5-40 nucleotides. In some embodiments, the UMI comprises 5-30 nucleotides. In some embodiments, the UMI comprises 5-25 nucleotides. In some embodiments, the UMI comprises 5, 6, 7, 8, 9, 10, 11, 12, 13, 14, 15, 16, 17, 18, 19, 20, 21, 22, 23, 24, or 25 nucleotides. In some embodiments, the UMI comprises 17 nucleotides.

In some embodiments, each capture oligonucleotide comprises a spatial barcode. The spatial barcode may be an oligonucleotide of any suitable length. In some embodiments, the spatial barcode sequence of a capture oligonucleotide depends on the spatial barcode sequence of the surface probe to which the capture oligonucleotide was hybridized. For example, in some embodiments, the spatial barcode comprises 10-100 nucleotides. In some embodiments, the spatial barcode comprises 10-50 nucleotides. In some embodiments, the spatial barcode comprises 10-30 nucleotides. In some embodiments, the spatial barcode comprises 15-25 nucleotides. In some embodiments, the spatial barcode comprises 10, 11, 12, 13, 14, 15, 16, 17, 18, 19, 20, 21, 22, 23, 24, 25, 26, 27, 28, 29, or 30 nucleotides. In some embodiments, the spatial barcode comprises 18 nucleotides.

In some embodiments, each capture oligonucleotide comprises at least one anchor sequence. In some embodiments, the capture oligonucleotide comprises a first anchor sequence and a second anchor sequence. In some embodiments, the first anchor sequence is proximal to the support. In some embodiments, the first anchor sequence and the second anchor sequence are separated by the spatial barcode. The first anchor sequence and/or the second anchor sequence may comprise any suitable number of contiguous nucleotides.

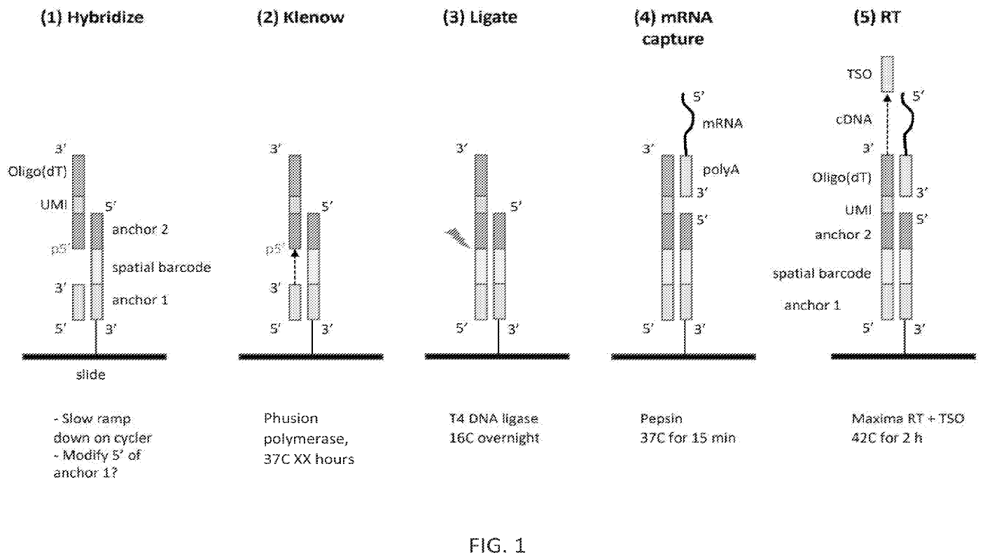

In some embodiments, the plurality of capture oligonucleotides within each spot are immobilized on the support. The capture oligonucleotides may be immobilized on the support by hybridization with surface probes. For example, a support comprising an array of surface probes may be provided, and the capture oligonucleotides described herein may be immobilized to the surface probes. For example, a suitable method for generating the systems described herein is shown in FIG. 1.

In some embodiments, the systems described herein are generated by providing a support comprising an array of surface probes. In some aspects, provided herein are methods of producing systems for spatial detection of nucleic acid in a tissue sample. In some embodiments, methods for producing a system for spatial detection of nucleic acid in a tissue sample comprise providing a support comprising an array of surface probes and performing reactions directly on the support, thereby creating a system comprising a plurality of spots, each spot containing a plurality of capture probes. In some embodiments, each surface probe on the support comprises a first anchor sequence, a spatial barcode, and a second anchor sequence. In some embodiments, the method comprises hybridizing a first complementary oligonucleotide to the first anchor sequence of each surface probe and hybridizing a second complementary oligonucleotide to the second anchor sequence of each surface probe. In some embodiments, the second oligonucleotide comprises a nucleic acid capture region (e.g. a capture domain) and a unique molecular identifier. In some embodiments, the method comprises performing an extension-ligation reaction on the support. The extension-ligation reaction performed directly on the support is also referred to herein as an “on-slide” reaction. In some embodiments, the extension-ligation reaction comprises extending the first complementary nucleotide with a sequence complementary to the spatial barcode of the surface probe, and ligating the extended first complementary nucleotide and the second complementary nucleotide together to form a contiguous capture oligonucleotide. The contiguous capture oligonucleotide comprises a nucleic acid capture region, a unique molecular identifier, a first anchor sequence, a spatial barcode, and a second anchor sequence.

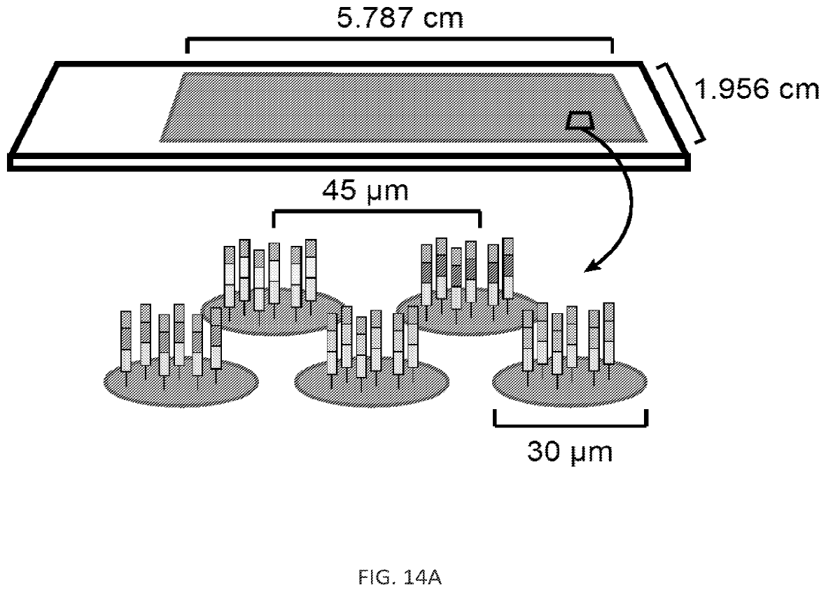

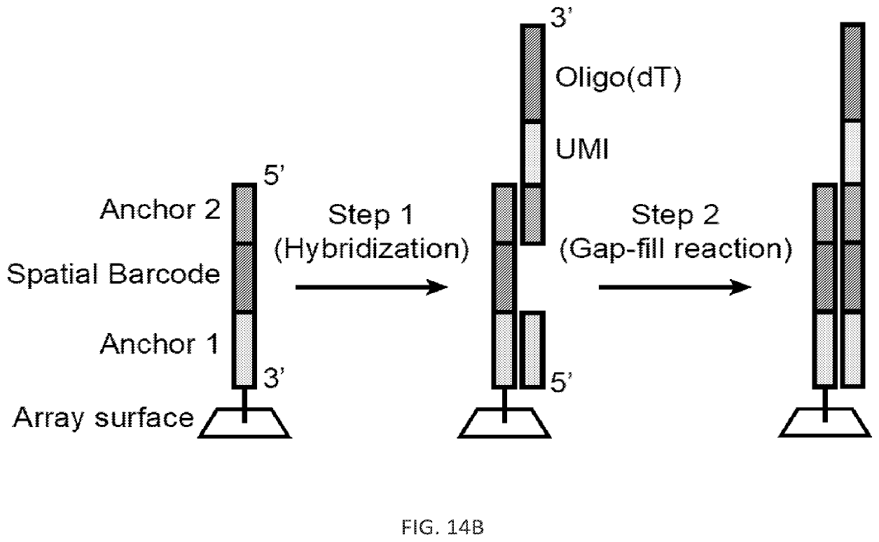

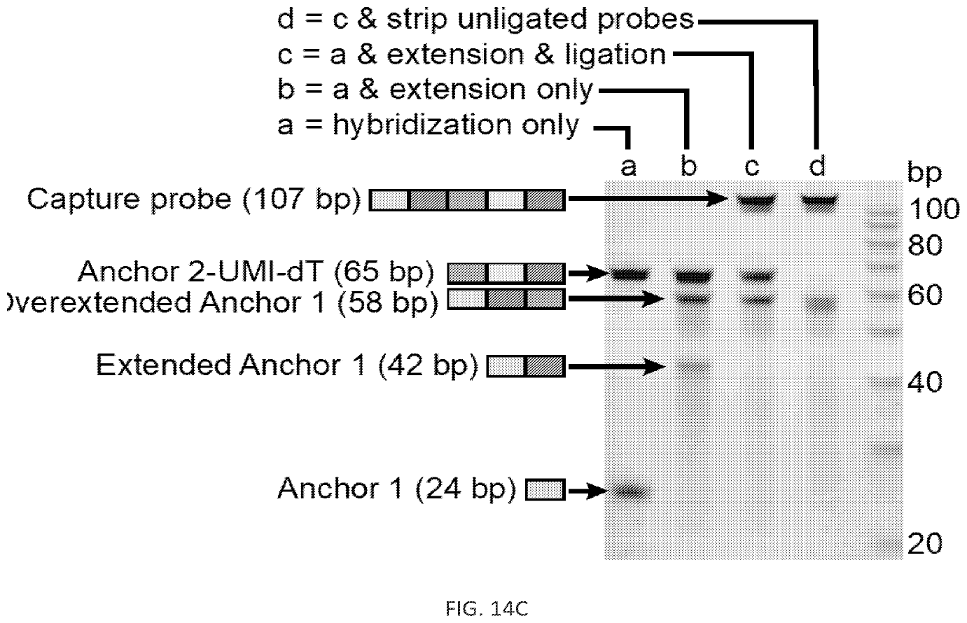

In some embodiments, each surface probe comprises a first anchor sequence, a spatial barcode, and a second anchor sequence. A first complementary nucleotide may be hybridized to the first anchor sequence of a surface probe (for example, as shown in FIG. 1, step 1). For example, the first complementary nucleotide may comprise a first anchor sequence that is complementary to the first anchor sequence of the surface probe. A second complementary nucleotide may be hybridized to the second anchor sequence of a surface probe (for example, as shown in FIG. 1., step 1, and FIG. 14B). The second complementary nucleotide may comprise an anchor sequence that is complementary to the second anchor sequence of the surface probe, and may further comprise a nucleic acid capture region and a unique molecular identifier. The first complementary nucleotide may be extended with a sequence complementary to the spatial barcode of the surface probe (as shown in FIG. 1., step 2, and FIG. 14B), such that the first complementary nucleotide and the second complementary nucleotide form a contiguous capture oligonucleotide. The capture oligonucleotide comprises a nucleic acid capture region, a unique molecular identifier, a first anchor sequence, a spatial barcode, and a second anchor sequence, as described herein.

In some embodiments, the first anchor sequence of a surface probe (e.g. a surface probe immobilized on the support) hybridizes to an anchor sequence of a first complementary oligonucleotide that ultimately forms a capture probe upon extension to be joined with a second complementary oligonucleotide, as described above. Any suitable size and base composition of the first anchor sequence of a surface probe may be used. In some embodiments, the first anchor sequence of a surface probe is selected for compatibility with a desired sequencing primer or system. In some embodiments, the first anchor sequence comprises 10-30 bases. In some embodiments, the first anchor sequence comprises 10, 11, 12, 13, 14, 15, 16, 17, 18, 19, 20, 21, 22, 23, 24, 25, 26, 27, 28, 29, or 30 bases. In some embodiments, the first anchor sequence comprises 24 bases.

In some embodiments, the second anchor sequence of a surface probe hybridizes to an anchor sequence of a second complementary oligonucleotide that ultimately forms a capture probe, as described above. Any suitable size and composition of the second anchor sequence of a surface probe may be used. In some embodiments, the second anchor sequence comprises 10-30 bases. In some embodiments, the second anchor sequence comprises 10, 11, 12, 13, 14, 15, 16, 17, 18, 19, 20, 21, 22, 23, 24, 25, 26, 27, 28, 29, or 30 bases. In some embodiments, the second anchor sequence comprises 16 bases.

In some embodiments, the spatial barcode sequence of a surface probe (and thus the spatial barcode sequence of a capture probe generated following hybridization to the surface probe) comprises 10-100 bases. In some embodiments, the spatial barcode sequence of a surface probe comprises 10-50 bases. In some embodiments, the spatial barcode sequence of a surface probe comprises 10-30 nucleotides. In some embodiments, the spatial barcode sequence of a surface probe comprises 15-25 nucleotides. In some embodiments, the spatial barcode sequence of a surface probe comprises 10, 11, 12, 13, 14, 15, 16, 17, 18, 19, 20, 21, 22, 23, 24, 25, 26, 27, 28, 29, or 30 nucleotides. In some embodiments, the spatial barcode sequence of a surface probe comprises 18 nucleotides.

In some embodiments, the first anchor sequence, the spatial barcode sequence, and the second anchor sequence are designed to avoid self-dimerization. Suitable commercial packages may be used to optimize the design of the first anchor sequence, spatial barcode sequence, and second anchor sequence of the surface probes. For example, the commercial package DNABarcodes²⁶ may be used for design of optimal surface probe sequences (e.g. first anchor sequence, spatial barcode sequence, and second anchor sequence).

In some embodiments, for each surface probe, the first anchor sequence, the second anchor sequence, and the spatial barcode each comprise 10-30 nucleotides. In some embodiments, for each surface probe the first anchor sequence comprises 20-30 nucleotides, the second anchor sequence comprises 10-20 nucleotides, and the spatial barcode comprises 15-25 nucleotides. For example, in some embodiments for each surface probe the first anchor sequence, the second anchor sequence, and the spatial barcode each comprise 10, 11, 12, 13, 14, 15, 16, 17, 18, 19, 20, 21, 22, 23, 24, 25, 26, 27, 28, 29, or 30 nucleotides. In some embodiments, for each surface probe the first anchor sequence comprises 24 nucleotides, the second anchor sequence comprises 16 nucleotides, and the spatial barcode comprises 18 nucleotides.

In some embodiments, for each surface probe the second anchor sequence comprises 40% to 60% guanosine and/or cytosine (G/C) bases. For example, in some embodiments for each surface probe the second anchor sequence comprises about 40%, about 42.5%, about 45%, about 47.5%, about 50%, about 52.5%, about 55%, about 57.5%, or about 60% G/C bases. In some embodiments, the distribution of G/C bases is substantially uniform throughout the second anchor sequence. In some embodiments, the distribution of G/C bases is biased towards the 5′ end of the second anchor sequence. In some embodiments, the distribution of G/C bases is biased towards the 3′ end of the second anchor sequence.

In some embodiments, the nucleic acid capture region comprises at least 10 deoxythymidine residues.

In some embodiments, performing the extension ligation reaction on the support comprises adding a DNA polymerase and a DNA ligase to the support under conditions such that the reverse complement of the spatial barcode sequence is synthesized and ligated to the first complementary nucleotide and to the second complementary nucleotide, thereby forming the continuous capture probe.

In some aspects, provided herein are methods for spatial detection of nucleic acid in a tissue sample. The methods comprise contacting the tissue sample with a system as described herein. In some embodiments, the nucleic acid is RNA. In some embodiments, provided herein are methods for spatial detection of RNA in a tissue sample.

In some embodiments, provided herein is a method for spatial detection of RNA in a tissue sample comprising contacting a system described herein with a tissue sample, such that RNA within the tissue sample to binds to the capture oligonucleotides. For example, in some embodiments the method comprises contacting a system as described herein with a tissue sample, such that RNA within the tissue sample hybridizes to the capture domain of a capture probe, which capture probe is bound to the support (e.g. array surface), such as by hybridization to a surface probe immobilized on the surface of the support. For example, the poly-A tail of RNA molecules (e.g. mRNA) may bind to the poly-T (or functionally equivalent) domain of the capture oligonucleotides. This is shown in FIG. 1, step 4.

In some embodiments, the method further comprises reverse-transcribing the bound RNA to generate cDNA. In some embodiments, the method further comprises sequencing the cDNA. For example, in some embodiments the method comprises capturing the cDNA, and sequencing the captured cDNA.

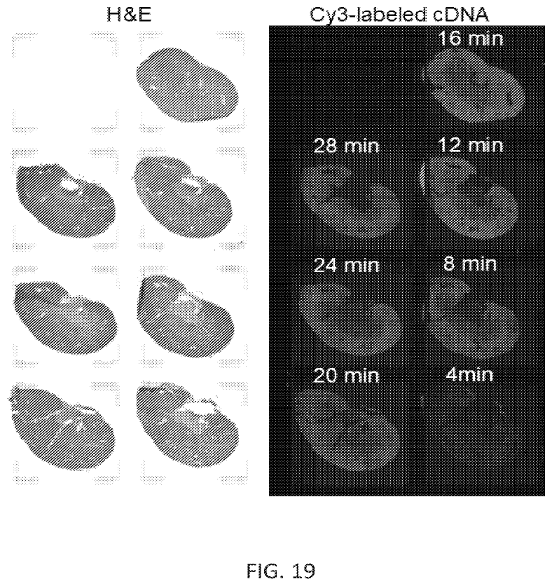

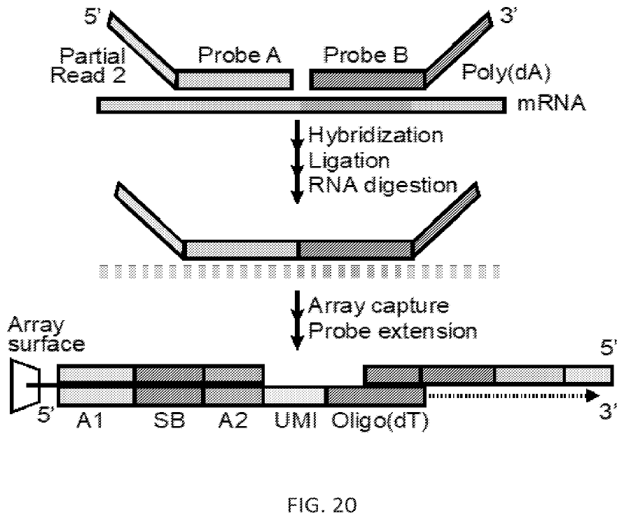

In some embodiments, methods for spatial detection of nucleic acid are performed using a hybridized tissue sample. In some embodiments, provided herein is a method for spatial detection of nucleic acid in a tissue sample comprising hybridizing the tissue sample with a suitable hybridization probe(s). In some embodiments, the hybridization probe(s) bind to target nucleic acid (e.g. target RNA) in the tissue sample, and also bind to the capture oligonucleotides of the array (e.g. the capture oligonucleotides bound to the surface of the array via hybridization to capture probes immobilized on the array surface). In some embodiments, the method for spatial detection of RNA in a tissue sample comprise hybridizing a first probe and a second probe to a target RNA sequence in a tissue sample. An exemplary embodiment involving a first probe and a second probe is shown, for example, in FIG. 20. In some embodiments, the first probe and the second probe each comprise an RNA binding region complementary to the target RNA sequence. In some embodiments, the method comprises ligating the RNA binding region of the first probe to the RNA binding region of the second probe, thereby forming an extended probe. The extended probe is hybridized to the target RNA sequence in the tissue sample and is therefore complementary to a portion of the target RNA sequence in the tissue sample. In some embodiments, the method comprises digesting RNA following ligation, thereby releasing the extended probe from the target RNA. In some embodiments, the method comprises contacting the tissue sample with a system as described herein, such that extended probes (which are complementary to the target RNA sequence) bind to the capture oligonucleotides on the support (e.g. on the surface of the array). In some embodiments, the first probe further comprises a capture oligonucleotide binding region complementary to the nucleic acid capture domain of a capture oligonucleotide, thereby allowing the extended probe to be captured by a capture oligonucleotide. In some embodiments, the second probe further comprises a sequencing handle to facilitate sequencing. In some embodiments, the method comprises reverse-transcribing the bound extended probes to generate cDNA, and sequencing the cDNA.

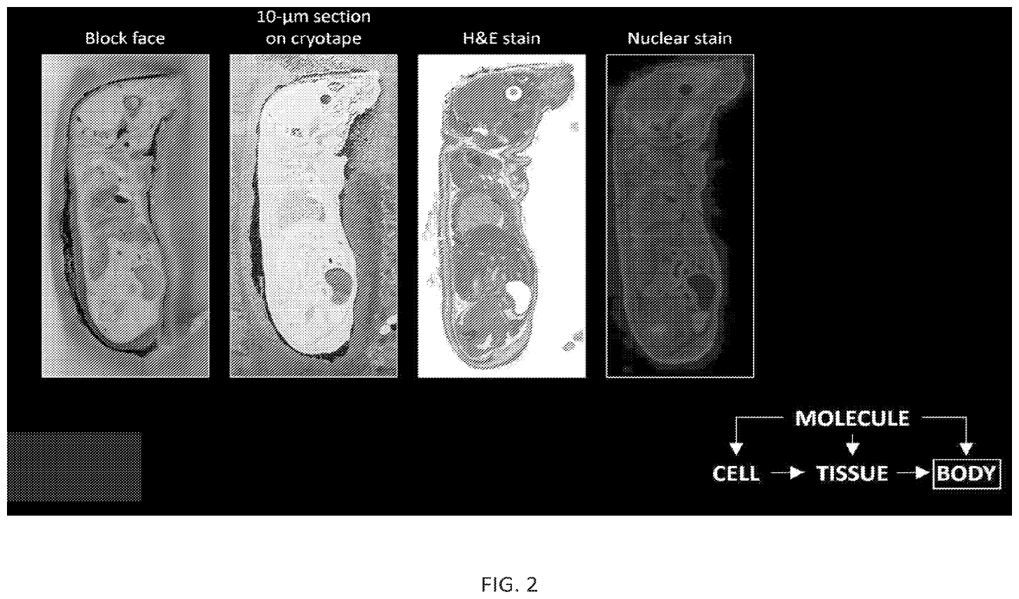

In some embodiments, provided herein are methods for spatial detection of RNA in a tissue sample having a surface area of at least 2 cm². In some embodiments, the method comprises obtaining a tissue sample having a surface area of at least 2 cm², wherein the tissue sample is stabilized on an adhesive film. In some embodiments, the tissue has a surface area of at least 2 cm², at least 3 cm², at least 4 cm², at least 5 cm², at least 6 cm², at least 7 cm², at least 8 cm², at least 9 cm², at least 10 cm², at least 11 cm², at least 12 cm², at least 13 cm², at least 14 cm², at least 15 cm², at least 16 cm², at least 17 cm², at least 18 cm², at least 19 cm², or at least 20 cm². For example, as shown in FIG. 2, the systems and methods described herein enable detection of spatial location of nucleic acid in a slice (in this case, a sagittal slice) corresponding to the entire body of a mouse.

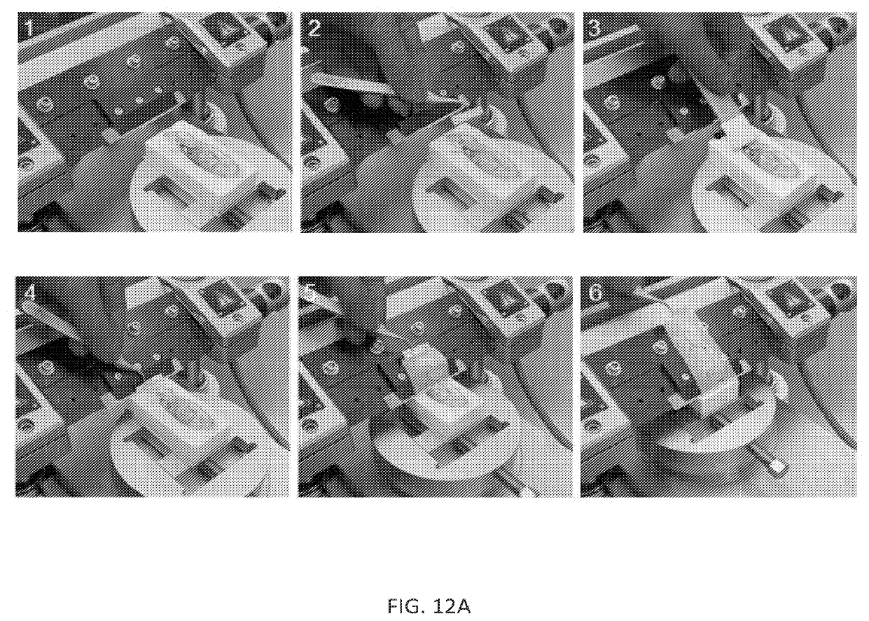

An exemplary method for stabilizing a tissue sample (e.g. a tissue slice) on an adhesive film is shown in FIG. 12. In some embodiments, the tissue is stabilized on the adhesive film by applying the adhesive film to an exposed surface of the tissue sample (e.g. on top of the tissue sample) and cutting a tissue slice using a suitable blade, thus creating a tissue sample (e.g. a tissue slice) wherein one side of the tissue is exposed (e.g. the portion that was not covered by the adhesive film prior to slicing) and the other side of the tissue is stabilized on the adhesive film.

In some embodiments, the method comprises transferring the tissue sample from the adhesive film to a system (e.g. array) as described herein. In some embodiments, transferring the tissue sample comprises mounting the stabilized tissue sample on the support (e.g. on the surface of the array) and dissolving the adhesive film in hexane, thereby transferring the tissue sample from the adhesive film to the support. In some embodiments, the method comprises transferring the tissue sample from the adhesive film to a system described herein, such that RNA within the tissue sample to binds to the capture oligonucleotides on the support (e.g. on the surface of the array). In some embodiments, the method comprises reverse-transcribing the bound RNA to generate cDNA, and sequencing the cDNA. In some embodiments, the method further comprising correlating a spatial barcode for each sequenced cDNA molecule with the location of the spot on the support having a corresponding spatial barcode. In some embodiments, the method further comprising imaging the tissue and/or staining the tissue after transferring the tissue sample from the adhesive film to the support.

In some embodiments, the method comprises transferring the tissue from the adhesive film to a system described herein, and hybridizing the tissue sample with one or more hybridization probes. Suitable methods and hybridization probes are described above. For example, in some embodiments the method comprises hybridizing the tissue sample with a first probe and a second probe that bind to a target RNA sequence in the tissue sample. For example, in some embodiments the first probe and the second probe each comprise an RNA binding region complementary to the target RNA sequence. In some embodiments, the method further comprises ligating the RNA binding region of the first probe to the RNA binding region of the second probe, thereby forming an extended probe that is hybridized to the target RNA sequence in the tissue sample. In some embodiments, the method comprises permitting extended probes to bind to capture oligonucleotides on the on the support, reverse-transcribing the bound extended probes to generate cDNA; and sequencing the cDNA. In some embodiments, permitting extended probes to bind to capture oligonucleotides on the support comprises digesting RNA, thereby releasing the extended probes from the target RNA and permitting binding of the extended probes to the capture oligonucleotides of the array.

For any of the methods described herein. cDNA may be generated by reverse-transcription. The cDNA generated is complementary to the bound RNA, and is therefore indicative of the RNA present in in a given cell at the time the tissue sample was obtained from the source organism. As shown in FIG. 1, step 5, the sequence of the capture oligonucleotide is incorporated into the sequence of the synthesized cDNA. Accordingly, the spatial barcode and the UMI of the capture oligonucleotide is incorporated into the sequence of the cDNA strand.

Generally speaking, reverse transcription is performed by adding a reverse transcriptase and suitable additional reagents (e.g. dNTPS, buffers, RNAse inhibitors, etc.) and holding the mixture at a suitable temperature for a suitable duration of time. Any suitable reverse transcriptase enzyme may be used, such as M-MLV, MuLV, AMV, HIV, ArrayScript™, MultiScribe™, ThermoScript™, or Superscript® I, II, and III enzymes. Typically, reverse transcriptase reactions are performed between 37-55° C. The reaction time may be as little as 1, minute, or as much as 48 hours. In some embodiments, the reverse transcription reaction is carried out at 42° C. for about 2 hours. In some embodiments, a template switch oligonucleotide (TSO) may be added during reverse transcription. For example, a TSO may be used to add a common 5′ sequence to the cDNA molecules that may be used for subsequent cDNA amplification.

In some embodiments, the methods described herein further comprise sequencing the cDNA. In some embodiments, the method comprises sequencing the strand of cDNA generated directly from reverse transcription of the bound RNA. This strand is referred to as the “first strand” cDNA. In some embodiments, a strand of cDNA complementary to the first strand cDNA is generated, referred to as a “second strand” cDNA. In some embodiments, sequencing cDNA comprises sequencing the second strand and/or the first strand cDNA. In some embodiments, the method comprises releasing the cDNA from the support, and optionally amplifying the cDNA, prior to sequencing. Sequencing may be performed by any suitable method. Sequencing is generally performed using one or multiple amplification steps, such as polymerase chain reaction (PCR). In some embodiments, sequencing may be performed using next-generation sequencing methods, such as high-throughput sequencing, sequencing by synthesis (e.g. ILLUMINA technology), pyrosequencing, and the like.

In some embodiments, the methods described herein further comprise correlating the spatial barcode for each sequenced cDNA molecule with the location of the spot on the support having a corresponding spatial barcode. In some embodiments, the location of each spot on the support may be determined prior to contacting the tissue sample with the system. In some embodiments, the location of each spot on the support is known. For example, a map or layout of the support may be provided to a user of the system, such that the user knows the spatial location of each spot (e.g. the location of a given spatial barcode) on the support. Accordingly, following determining the sequence of a given cDNA molecule, the location of that molecule on the support may be correlated with the known location of a spot containing the corresponding spatial barcode. Furthermore, the location of a corresponding nucleic acid on the tissue sample can be determined. For example, the location on the support comprising a given spatial barcode can be aligned with a given area of the tissue that was in contact with that location on the support during cDNA synthesis. Accordingly, the sequence of the spatial barcode of a given sequenced cDNA molecule can be correlated with a corresponding spatial barcode on the support, the location of which can be used to determine a precise area of tissue that was in contact with that location on the support. Accordingly, RNA expression (e.g. mRNA expression, indicative of gene expression) in a specific location of a tissue sample can be determined.

In some embodiments, the methods described herein further comprise imaging and/or staining the tissue. For example, the tissue may be imaged and/or stained before and/or after contacting the tissue with the support. Imaging and/or staining the tissue may assist in determining the spatial location of a given nucleic acid within the tissue. For example, imaging and/or staining the tissue may assist in correlating the spot on the support having a given spatial barcode (e.g. a spatial barcode corresponding to a given sequenced cDNA molecule) with a location on the tissue that was in contact with that spot on the support during cDNA synthesis. As another example, imaging and/or staining the tissue may assist in highlighting various regions within the organism itself. For example, as shown in FIG. 2., H&E stain may be used to highlight various compartments (e.g. organs) within a mouse slice, which may be used to further assist in determining spatial location of nucleic acid within each organ. In some embodiments, imaging and/or staining the tissue may be conducted after transferring the adhesive film-stabilized tissue from the adhesive film to the support.

The methods and substrates described herein may be used with any suitable tissue sample. The tissue may be fresh or frozen. In some embodiments, the tissue is a fresh-frozen tissue. The term “fresh-frozen” or “fresh frozen” when used in reference to a tissue refers to a tissue that was frozen after being obtained, without any fixation steps in-between. In some embodiments, the tissue may be fixed (e.g. formalin fixed). In some embodiments, the methods and systems described herein enable spatial detection of nucleic acid in a larger tissue sample than possible using previous spatial transcriptomics systems. For example, the tissue may have a surface area of at least 0.5 cm². For example, the tissue may have a surface area of at least 0.5 cm², 1 cm², 2 cm², 3 cm², 4 cm², 5 cm², 6 cm², 7 cm², 8 cm², 9 cm², 10 cm², 11 cm², 12 cm², 13 cm², 14 cm², 15 cm², 16 cm², 17 cm², 18 cm², 19 cm², or 20 cm². For example, as shown in FIG. 2, the systems and methods described herein enable detection of spatial location of nucleic acid in a slice (in this case, a sagittal slice) corresponding to the entire body of a mouse.

In some embodiments, the tissue is contacted with the system in a specific manner to facilitate use of a large slice (e.g. at least 1 cm², 2 cm², 3 cm², 4 cm², 5 cm², 6 cm², 7 cm², 8 cm², 9 cm², 10 cm², 11 cm², 12 cm², 13 cm², 14 cm², 15 cm², 16 cm², 17 cm², 18 cm², 19 cm², or 20 cm²) without ripping, tearing, or other contortions that affect the integrity of the tissue sample itself. For example, the tissue may be frozen prior to contact with the system. For example, the tissue may be fixed in an appropriate medium (e.g. paraformaldehyde, formaldehyde, etc.). The medium may further comprise one or more reagents, such as sugars (e.g. sucrose) at an appropriate concentration (e.g. 1-20%). The fixed tissue may be frozen by any suitable method. In some embodiments, the fixed tissue is rapidly frozen, such as by using dry ice, hexane-dry ice, liquid nitrogen, etc. The tissue may be placed in an appropriate embedding medium. For example, in some embodiments the tissue is fixed, frozen, and subsequently placed in an embedding medium. In some embodiments, the embedding medium comprises SCEM. In some embodiments, the embedding medium comprises SCEM-L1. In some embodiments, the embedded medium containing the tissue is subsequently frozen. For example, the embedded tissue may be frozen again using dry ice, hexane-dry ice, liquid nitrogen, etc.

For any of the embodiments described herein, the tissue is sliced to an appropriate thickness. For example, the tissue may be sliced using a cryostat. Furthermore, the tissue may be stabilized by use of an adhesive film. For example, an adhesive film may be applied to the exposed surface of the tissue prior to slicing. Following application of the adhesive film to the exposed surface, the tissue is sliced, such that a single slice is produced wherein one surface of the tissue is in contact with the adhesive film and the other surface of the tissue is not (e.g. in other words, only one surface of the tissue is exposed). The adhesive film-stabilized tissue may subsequently be transferred onto the oligonucleotide array. The adhesive film may be removed from the tissue prior to performing spatial transcriptomics measurements.

In some embodiments, the tissue sample is an FFPE tissue sample, and the method further comprises deparaffinizing the tissue sample and decrosslinking RNA in the tissue sample prior to contacting the tissue sample with a system as described herein. In some embodiments, the tissue sample is an FFPE tissue sample, and the method comprises deparaffinizing the tissue sample and decrosslinking RNA in the tissue sample prior to hybridizing the first probe and the second probe to the target RNA sequence. Suitable deparaffinization techniques include, for example, xylene washes. Suitable decrosslinking techniques include, for example, incubation at suitable temperatures with a chelating buffer.

In some aspects, provided herein are kits. In some embodiments, provided herein are kits for use in methods of spatial detection of nucleic acid in a tissue sample. In some embodiments, the kit comprises a system as described herein. For example, in some embodiments the kit comprises a support containing a plurality of spots immobilized on the support, wherein each spot comprises a plurality of capture oligonucleotides. In some embodiments, each capture oligonucleotide comprises a nucleic acid capture region, a unique molecular identifier, a first anchor sequence, and a spatial barcode. In some embodiments, each capture oligonucleotide further comprises a second anchor sequence. In some embodiments, the 5′ end of each capture oligonucleotide is proximal to the support, and the 3′ end of each capture oligonucleotide is furthest away from the support. In some embodiments, each capture oligonucleotide comprises, from 5′ end to 3′ end, a first anchor sequence, a spatial barcode, a second anchor sequence, a unique molecular identifier, and a nucleic acid capture region. In some embodiments, the capture oligonucleotides are immobilized on the support by hybridization to surface probes, which are immobilized on the surface of the substrate.

In some embodiments, the kit comprises a means for preparing a system by a method as described herein. For example, in some embodiments, the kit comprises a support comprising an array of surface probes. In some embodiments, each surface probe comprises a first anchor sequence, a spatial barcode, and a second anchor sequence. The surface probes may be immobilized on the support. In some embodiments, such a kit may be used to generate a system for spatial detection of nucleic acid as described herein. For example, a first complementary nucleotide may be hybridized to the first anchor sequence of a surface probe (for example, as shown in FIG. 1, step 1, and FIG. 14B). For example, the first complementary nucleotide may comprise a first anchor sequence that is complementary to the first anchor sequence of the surface probe. The first complementary nucleotide may be a part of a kit, or may be designed separately. In some embodiments, a second complementary nucleotide may be hybridized to the second anchor sequence of a surface probe (for example, as shown in FIG. 1., step 1, and FIG. 14B). The second complementary nucleotide may comprise an anchor sequence that is complementary to the second anchor sequence of the surface probe, and may further comprise a nucleic acid capture region and a unique molecular identifier. The second complementary nucleotide may be provided with the kit, or may be designed separately. The first complementary nucleotide may be extended with a sequence complementary to the spatial barcode of the surface probe (as shown in FIG. 1., step 2, and FIG. 14B), such that the first complementary nucleotide and the second complementary nucleotide form a contiguous capture oligonucleotide. The capture oligonucleotide comprises a nucleic acid capture region, a unique molecular identifier, a first anchor sequence, a spatial barcode, and a second anchor sequence, as described herein. The reagents necessary for such an extension reaction (e.g. a DNA polymerase, a DNA ligase) may be provided in the kit or may be selected/obtained separately.

In some embodiments, the kit further comprises additional reagents for spatial detection of nucleic acid in a tissue sample. For example, the kit may further comprise additional reagents for generation of cDNA, imaging of the tissue sample, for staining the tissue sample, and/or sequencing of cDNA. For example, the kit may further comprise enzymes (e.g. reverse transcriptases, ligases, etc.), dNTPs, buffers, RNAse inhibitors, primers, probes, labels (e.g. fluorescent dyes), and the like. Components of the kits may be physically packaged together or separately. The kits can also comprise instructions for using the components of the kit. Instructions can be supplied with the kit or as a separate component. Instructions may be in paper form, or an electronic form. For example, instructions may be provided on a computer readable memory device or downloaded from an internet website, or as recorded presentation.

In some embodiments, further provided herein is an electronic system for executing one or more steps in a method for determining the spatial location of nucleic acid in a tissue sample as described herein. In some embodiments, the electronic system comprises software. In some embodiments, the software contains instructions for performing one or more steps in a method described herein. For example, software may be designed to execute a program for generating cDNA, imaging tissue, performing PCR, performing sequencing, and the like. In some embodiments, the electronic system includes a memory for storing data collected during one or more steps in a method as described herein. For example, the memory may store sequencing and/or imaging data collected by a method as described herein. In some embodiments, the electronic system includes a computer (e.g., a controller), which may comprise the software and/or memory component.

### EXAMPLES

### Example 1

Agilent G3 microarray was customized and purchased from Agilent Technologies. The array featured a plurality of custom designed surface probes. Each surface probe comprises a first anchor sequence, a spatial barcode, and a second anchor sequence. Surface probes were immobilized to the surface of a solid support, such that the first anchor region was tethered to the support. The location of each cluster of surface probes, along with the corresponding spatial barcode for each cluster, was known.

Complementary nucleotides were hybridized to the surface probes as shown in FIG. 1. A first complementary nucleotide was hybridized to the first anchor sequence of each surface probe (FIG. 1, step 1). The first complementary nucleotide comprised a first anchor sequence that is complementary to the first anchor sequence of the surface probe. A second complementary nucleotide was hybridized to the second anchor sequence of a surface probe (as shown in FIG. 1., step 1). The second complementary nucleotide comprises an anchor sequence that is complementary to the second anchor sequence of the surface probe, a nucleic acid capture region, and a unique molecular identifier. The first complementary nucleotide was extended with a sequence complementary to the spatial barcode of the surface probe (as shown in FIG. 1., step 2), such that the first complementary nucleotide and the second complementary nucleotide formed a contiguous capture oligonucleotide. The capture oligonucleotide comprises, from 5′ end (closest to the support) to 3′ end (farthest from the support): a first anchor sequence, a spatial barcode, a second anchor sequence, a unique molecular identifier, and a nucleic acid capture region (e.g. an oligodT tail).

The resulting system comprised a plurality of spots, each spot containing a plurality of capture oligonucleotides. Each capture oligonucleotide within a given spot comprises the same spatial barcode. The spatial barcode for each spot is unique (i.e., no two spots comprise the same spatial barcode). Each capture oligonucleotide comprises a unique molecular identifier.

Tissue was placed upon the system, and the poly A tail of RNA (e.g. mRNA) binds to the polyT tail of the capture region of the capture oligonucleotides. cDNA can be synthesized as shown in FIG. 1, step 5., and the resulting cDNA can be sequenced. The sequence of a given cDNA can be correlated with a corresponding spatial barcode on the support to identify the location of the spot from which the cDNA originated. The location of this spot can be correlated with a precise location on the tissue sample that was in contact with that spot when the cDNA was synthesized. Accordingly, the precise spatial location of RNA expression (e.g. gene expression) in the tissue sample can be determined.

### Example 2

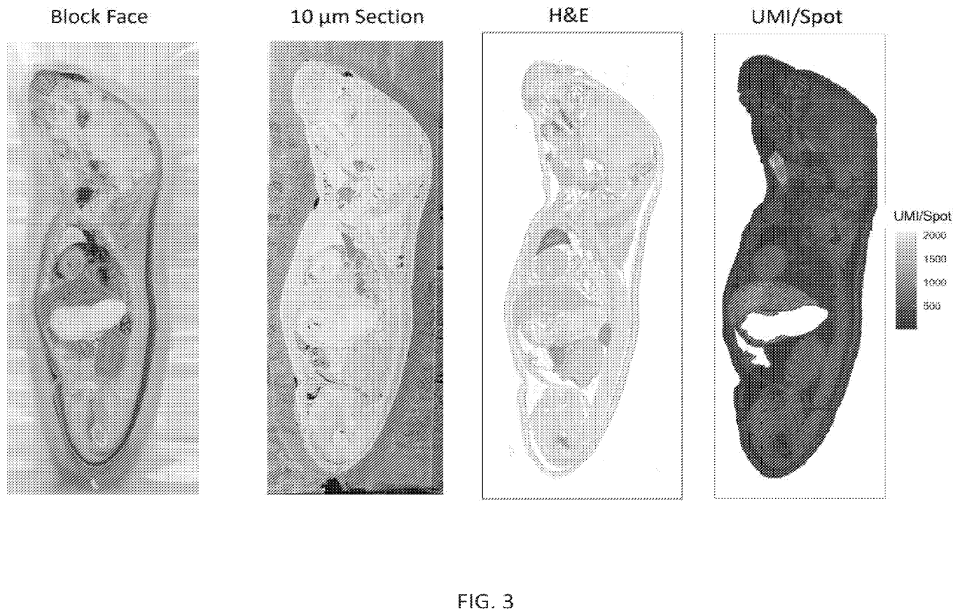

Sagittal slices were obtained from a whole mouse. The tissue was fixed, frozen, and embedded. An embedded tissue is shown in FIG. 3., “block face”. The embedded tissue was sliced using a cryostat. The tissue was stabilized by use of crytotape. Briefly, cryotape was applied to the exposed surface of the tissue prior to slicing. Following application of the cryotape to the exposed surface, the tissue was sliced to 10 μm thick, such that a single slice was produced wherein one surface of the tissue is in contact with the cryotape and the other surface of the tissue is not. The cryotape-stabilized tissue is shown in FIG. 3., “10 μm Section”. Histological images of sliced tissue were obtained by H&E staining to visualize structures within the slice.

Cryotape-stabilized slices were then transferred a system comprising a plurality of spots as described herein. The system was maintained under suitable hybridization conditions to allow the RNA within the slice to hybridize to capture probes. Following hybridization, reverse-transcription was performed to produce cDNA. The resulting cDNA was sequenced.

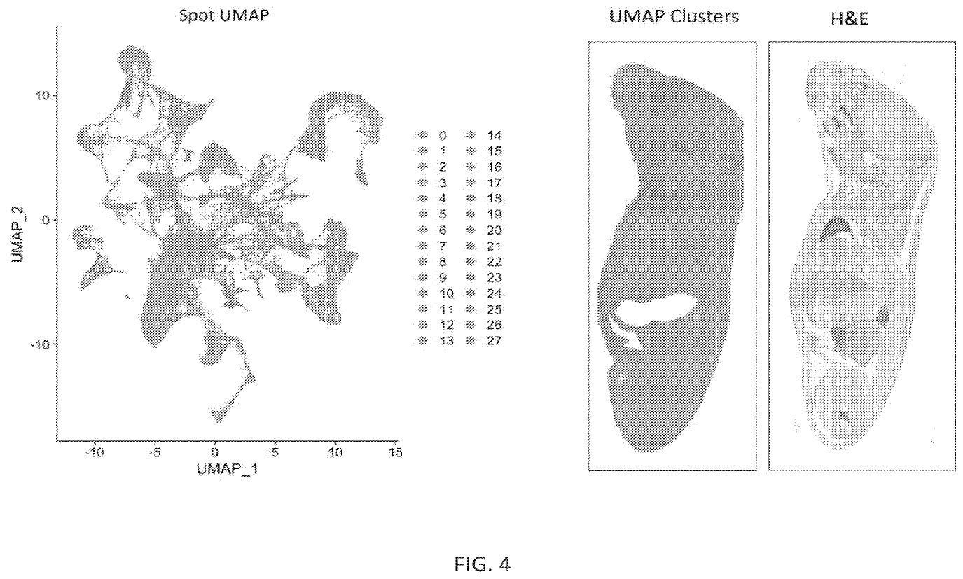

The number of unique molecular identifiers per spot, which is indicative of the number of capture probes per spot, was visualized (FIG. 3). UMAP plots were generated to show gene expression clusters within each spot. UMAP clusters were generated and compared to H&E staining to map clusters showing organ-specific gene expression (FIG. 4).

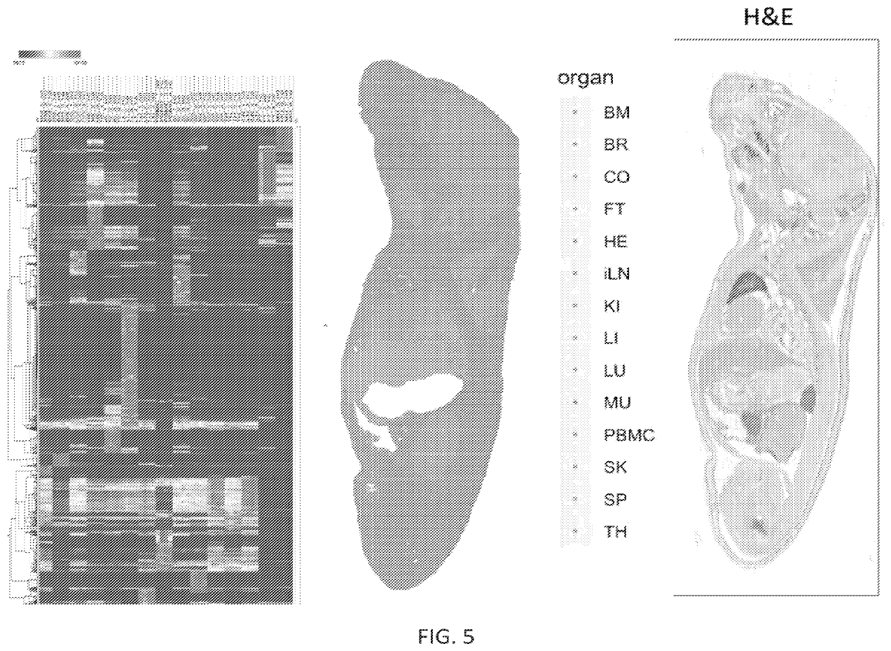

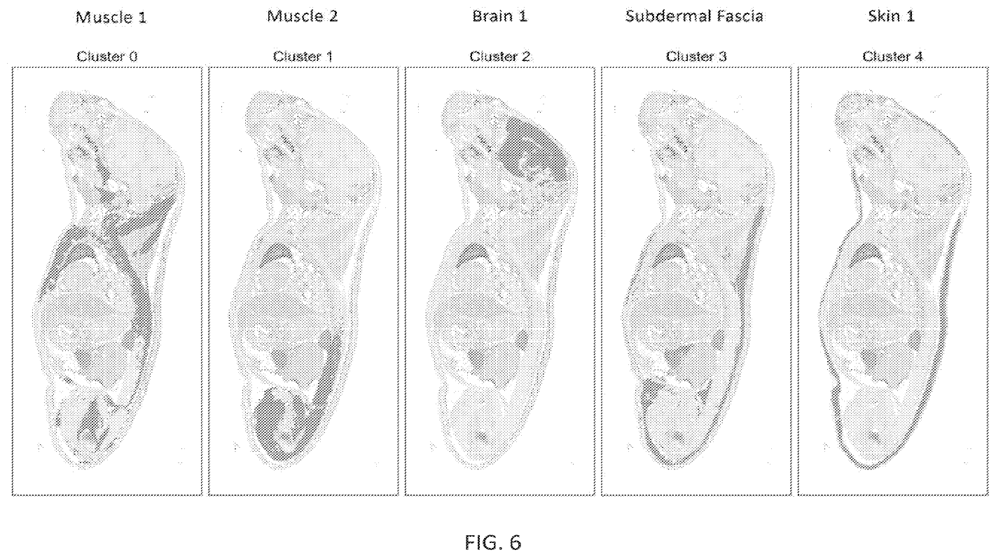

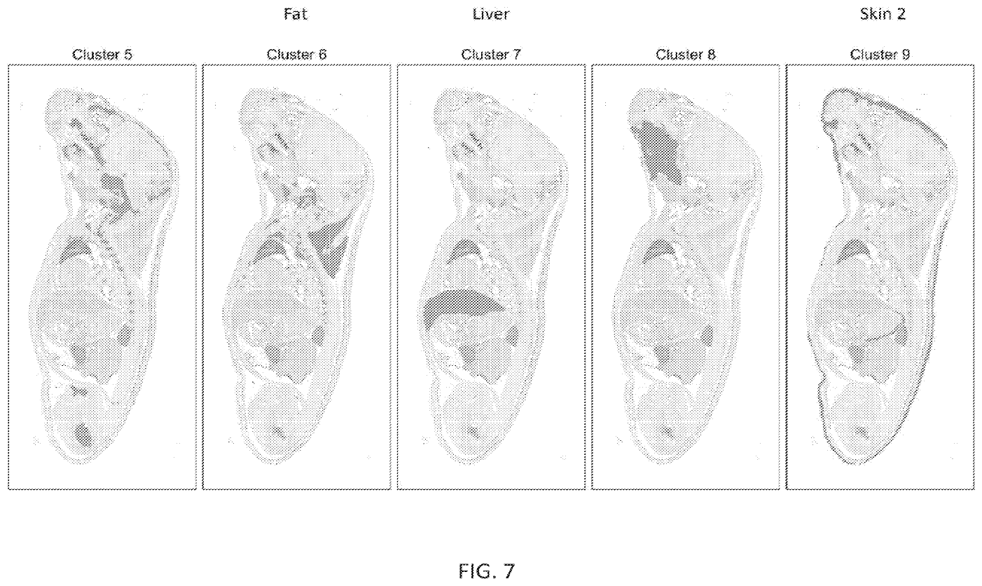

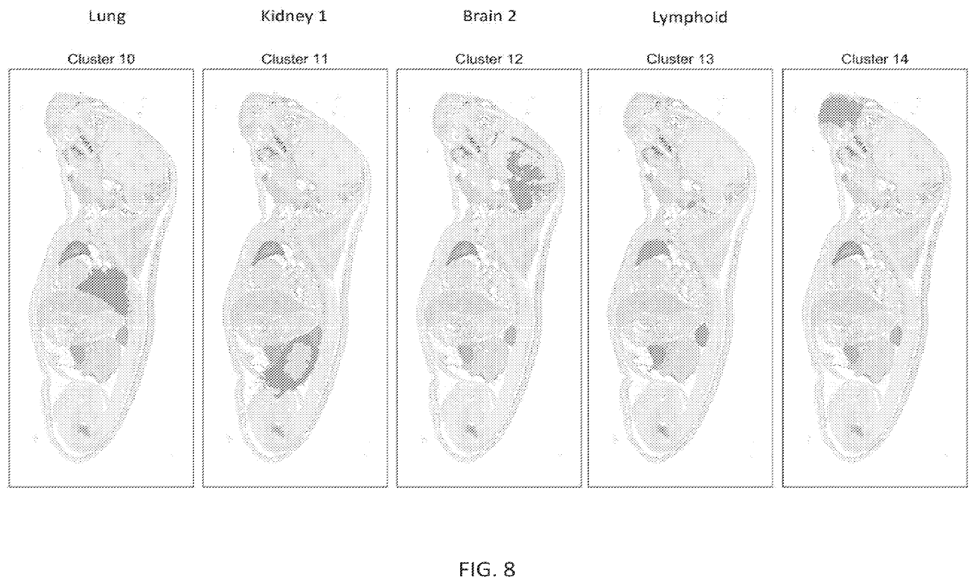

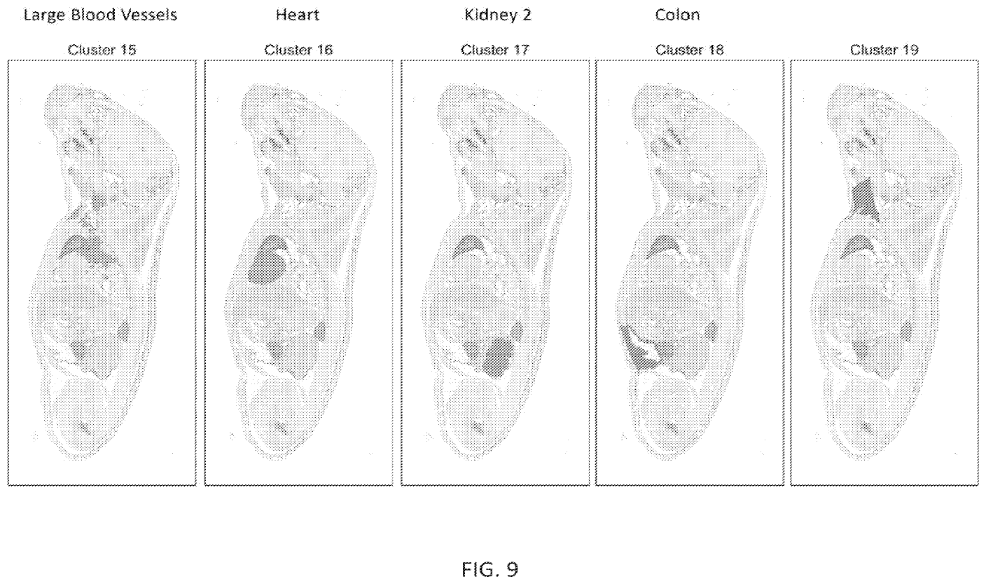

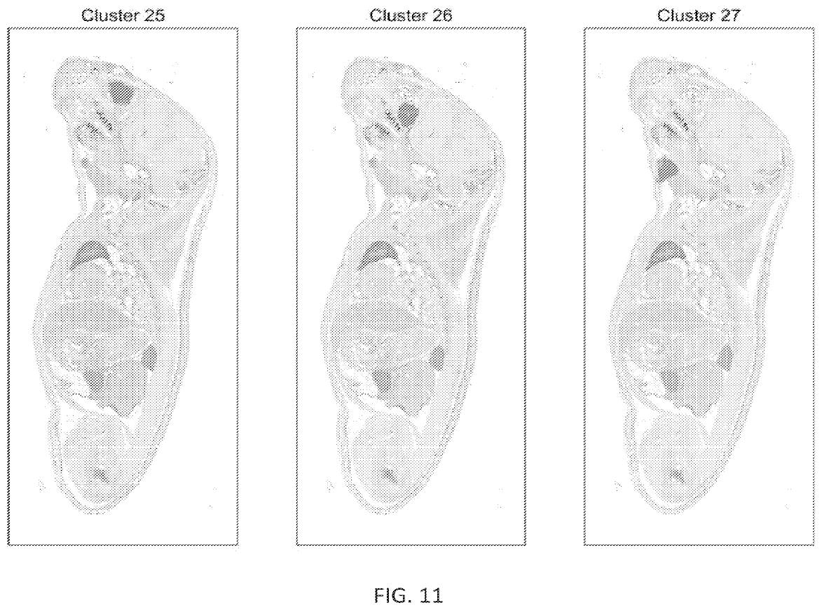

As shown in FIG. 5., the methods described herein successfully identified expression-based classification of organs using whole-tissue RNA-sequencing. Images of clusters of individual organs are shown in FIG. 6-11.

### Example 3

### Robust Methods for Preparation and Histological Sectioning of Small-to-Large Specimens

Current ST methods use conventional freeze-sectioning techniques of small (<1 cm²), mostly soft tissues. However, these sectioning techniques rely on cryomicrotomes that do not work for large or complex samples containing various tissue types such as whole-mouse sections. In addition, these sectioning techniques often result in tissue section damage, such as structural dislocation or abnormalities (e.g., tearing, folding), and are not amenable to robust serial sectioning. To circumvent these issues of reproducibility and scale across specimen types and sizes, various strategies were developed and used in the systems and methods described herein. First, the default blade holders and blade types of a cryomicrotome (Leica CM3050 S) and a cryomacrotome (Leica CM3600 XP) were modified and used to section standard (up to 1 cm²) and large-format (up to 12 cm²) specimens, respectively (FIG. 12A). Second, to avoid damage, maximize reproducibility, and enable serial sectioning, tissue sections were supported with pressure-sensitive adhesive film. By combining hardware improvements for cryo-micro/macro-tomes and a custom adhesive film, sectioning of small-to-large samples containing any tissue type was performed with high reproducibility and quality. For example, serial whole-mouse sections were successfully obtained while maintaining the finest histological structures (FIG. 12B-C). Notably, these sectioning methods are broadly applicable to any kind of samples from mouse and human but also fish, insects, or plants.

Transfer of Histological Sections from a Custom Adhesive Film to ST Arrays

To perform ST profiling on histological sections supported by the custom adhesive film, the section is first transferred to the ST array (i.e., glass slide carrying oligonucleotides). The use of adhesive film enables transfer of large-format sections (e.g., whole mount human organs or rodents), which typically cannot be obtained without the use of an adhesive film as support without extensive damage occurring to the tissue slice. In addition, the use of adhesive film enables the robust serial sectioning of small-to-large samples (e.g., 3D reconstruction of serial sections), which is not feasible with standard freeze-sectioning and manual transfer steps (e.g., forceps) without extensive section loss or damage.

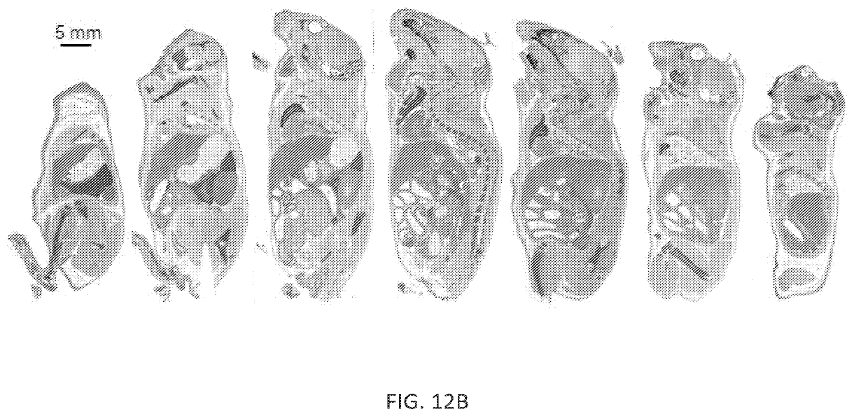

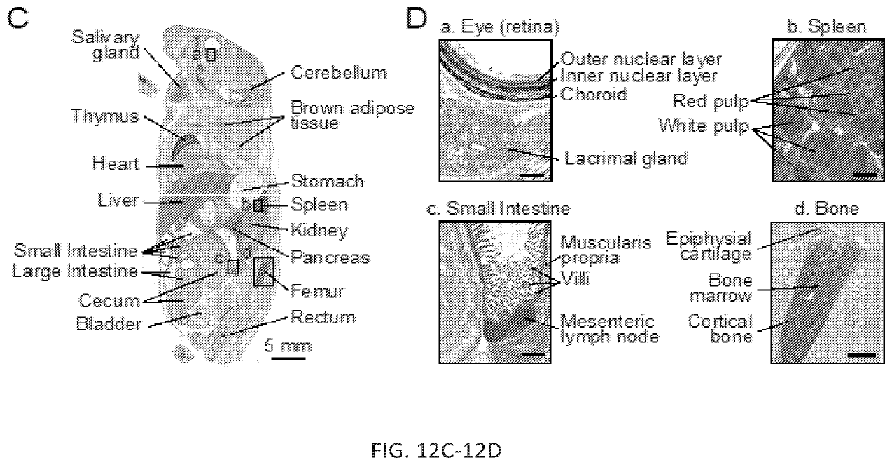

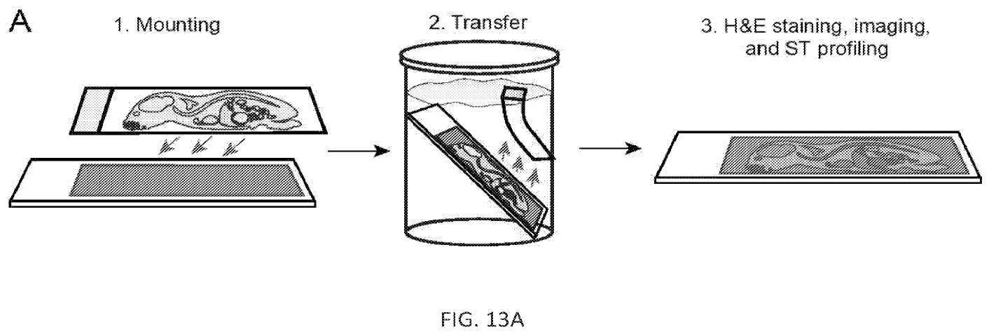

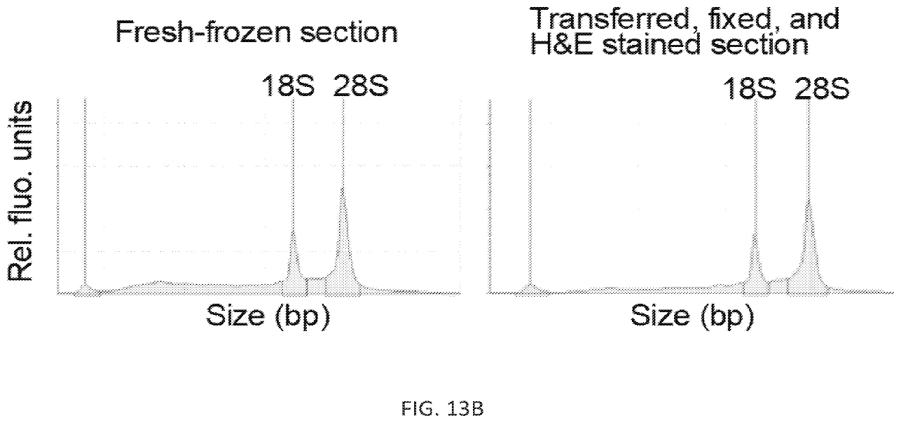

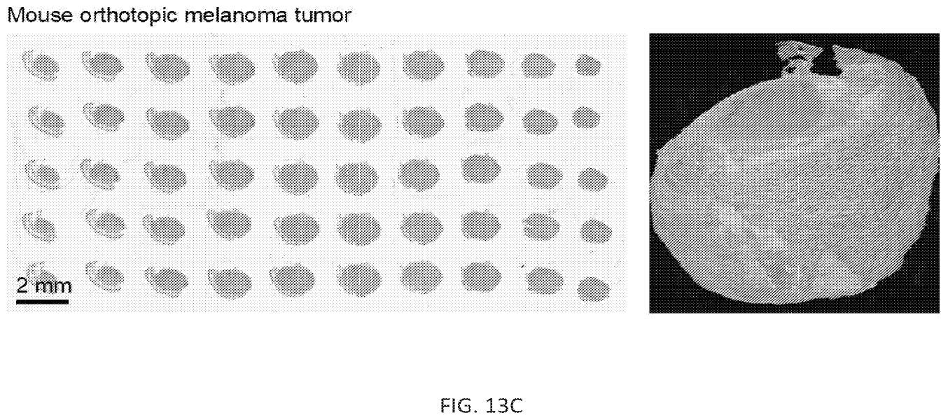

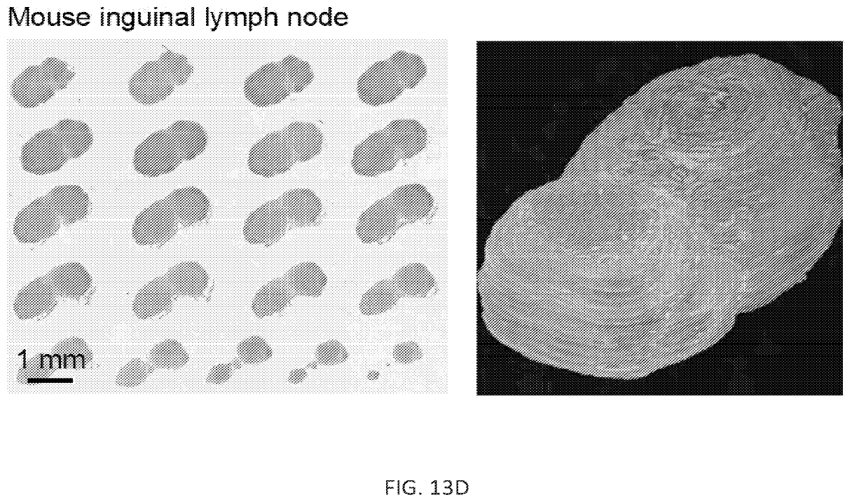

Fresh-frozen sections supported on an adhesive film (for example, obtained as shown in FIG. 2A) are transferred to glass slides by immersing samples into hexane at −20° C. to dissolve the adhesive material (FIG. 13A). After film removal, the transferred sections are fixed in methanol, stained with hematoxylin and eosin (H&E), imaged for histological analysis, and processed for custom ST profiling. The robustness of this transfer method in obtaining high-quality RNA (FIG. 13B) was demonstrated. Accordingly, the methods described herein enable 3D ST analysis by covering the entire volume of an organ using serial sectioning (FIG. 13C-D).

### Scalable Oligonucleotide Arrays for Spatial Transcriptomics (ST) Profiling

To enable scalable ST profiling, a large-format mRNA capture array was generated and employed. A commercially available oligonucleotide microarrays (Agilent) (FIG. 14A) was used in this example, although other suitable microarrays can be employed. These large-format ST arrays can be divided into multiple chambers using gaskets for standard microscopy slides to accommodate various experimental designs. Each spot on the array carries 3′-anchored oligonucleotides (up to 60-mer) (FIG. 14A). Custom arrays carrying two common sequences fixed across all spots (Anchor 1 and 2) and 18-mer spatial barcodes unique to each spot on the array (FIG. 4B) were generated. Arrays were first hybridized with two oligonucleotides: (i) a partial Illumina Read 1 sequence (Anchor 1), and (ii) a probe for mRNA capture containing the reverse complement of Anchor 2, unique molecular identifiers (UMIs) 18, and oligo (dT). Second, an on-slide extension-ligation (or “gap-fill”) reaction was performed using a DNA polymerase to synthesize the reverse complement of the spatial barcode in the 3′ end of the hybridized Anchor 1, and a DNA ligase to attach the newly synthesized barcode to the 5′ end of the capture probe (FIG. 14C). Third, arrays carrying fully assembled mRNA capture probes were processed to remove unligated probes (Anchor 2-UMI-dT) (FIG. 14C). Resulting arrays can be used as a scalable, cheap, and easy-to-adopt platform for ST profiling.

### Custom Arrays Outperform the Only Commercially Available ST Arrays.

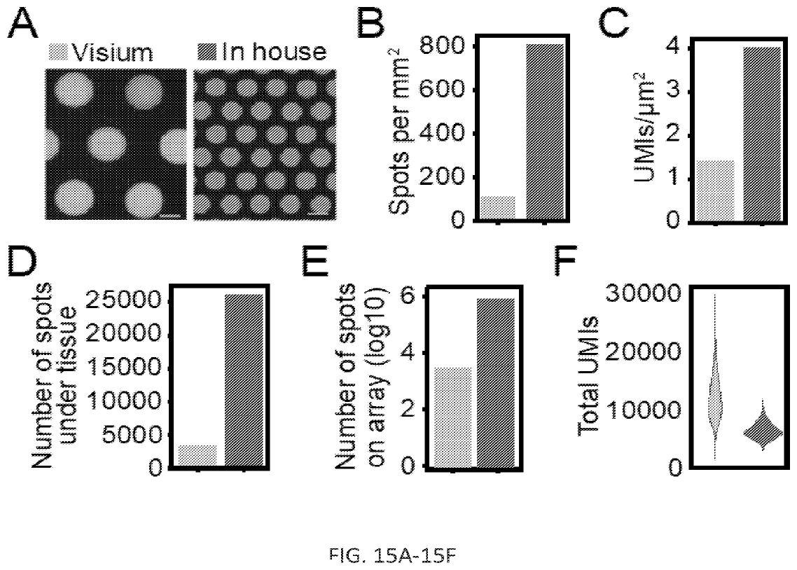

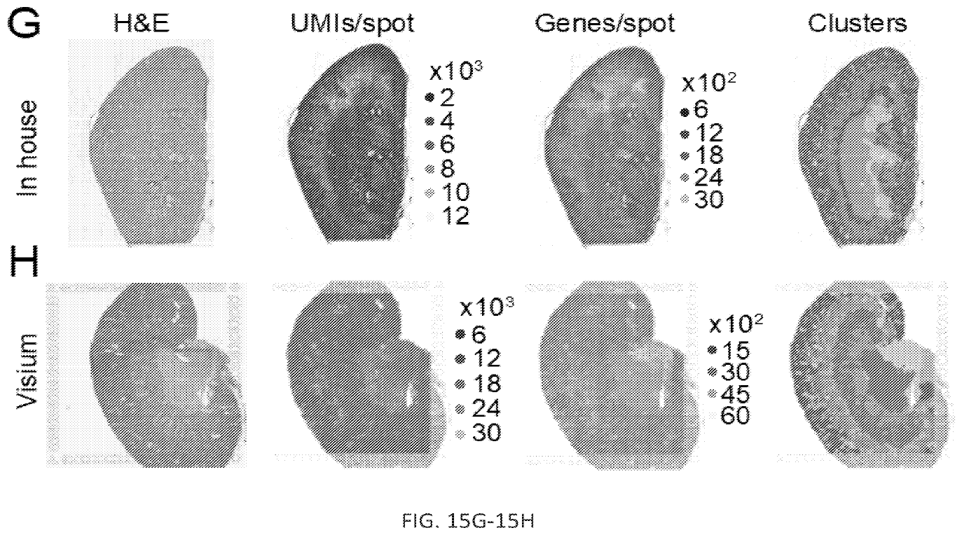

10-μm histological sections from mouse kidneys were used to benchmark the custom array described above against the commercial Visium kit (10× Genomics) using similar sequencing depth for both platforms. For ST using Visium, the manufacturer's experimental and computational pipelines were followed. For ST using the systems described herein, custom microarrays were built as described above (FIG. 14) and subdivided with gaskets (Grace Bio-labs) to delimit multiple, independent incubation chambers. Kidney sections were placed onto the custom, ST-ready arrays and permeabilized (15 min at 37° C. with 0.1% pepsin in 0.1M HCl) prior to in-tissue cDNA synthesis coupled with template switching¹⁹. After reverse transcription (RT), tissues were removed using proteinase K and full-length cDNAs were retrieved from the array (0.1M KOH), purified (Zymo column), and amplified by single-primer PCR. Amplified cDNAs were tagmented (Nextera kit, Illumina), enriched by index PCR, and sequenced (NextSeq550, Illumina). A computational pipeline was developed for: (i) raw read preprocessing using STARsolo²⁰ to generate UMI count matrices containing the coordinates for spots on the array; (ii) filtering data from spots that are not under the tissue by integrating the H&E image and ST data of the tissue section using custom Python scripts and published packages such as Scanpy²¹ and scikit-image²²; (iii) ST data clustering and differential expression analysis using Scanpy; and (iv) custom ST data visualizations using the Python library Seaborn²³. The custom arrays outperformed Visium in all metrics tested, including the overall density of spots on the array (˜8-fold) (FIG. 15A-B), the number of spots under the tissue (˜5-fold) (FIG. 15C), and UMIs per μm² (˜3-fold) (FIG. 15D). Clustering identified spatial areas matching known regions of the kidney for both Visium and the custom arrays (FIG. 15E-F), with a higher resolution achieved for the custom arrays given the higher spot density compared to Visium (FIG. 15A).

### Demonstration of Large Format ST Using Whole, Adult Mouse Sections

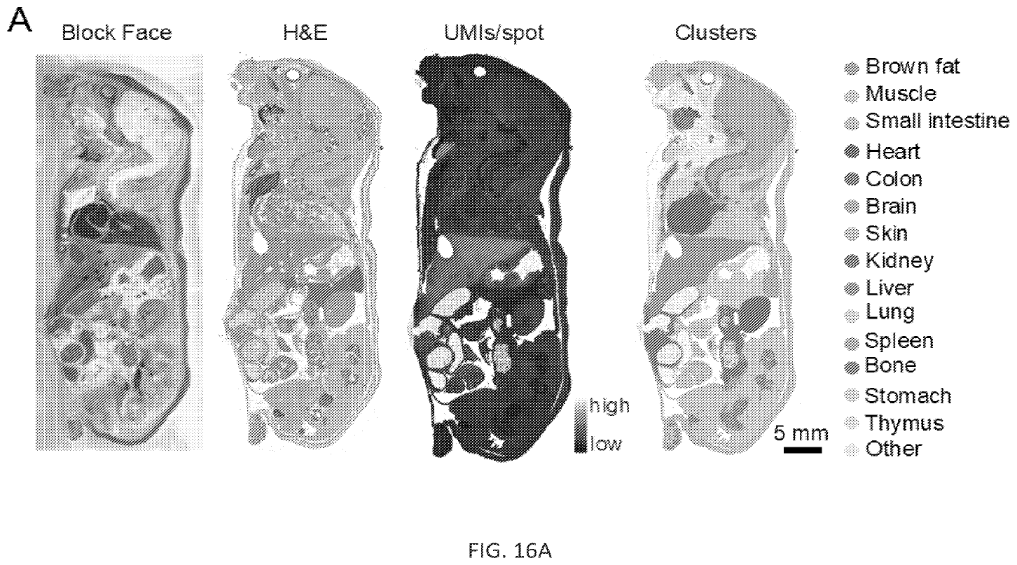

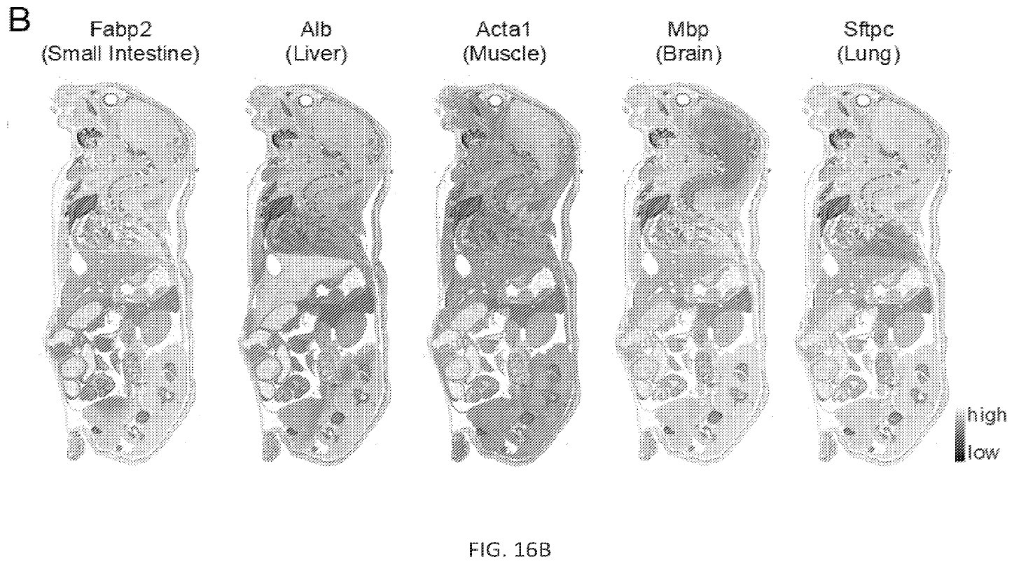

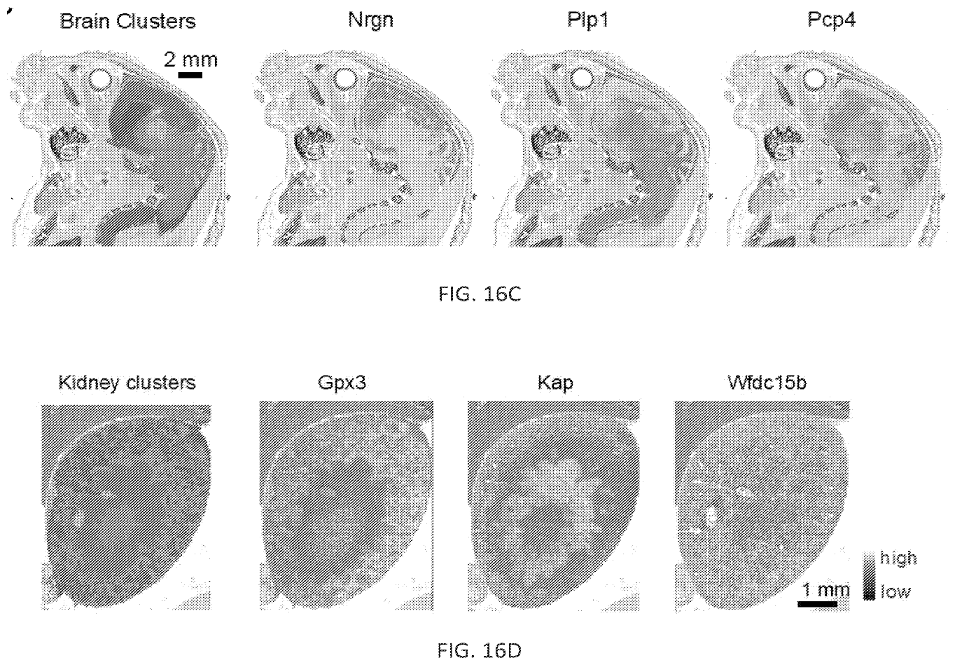

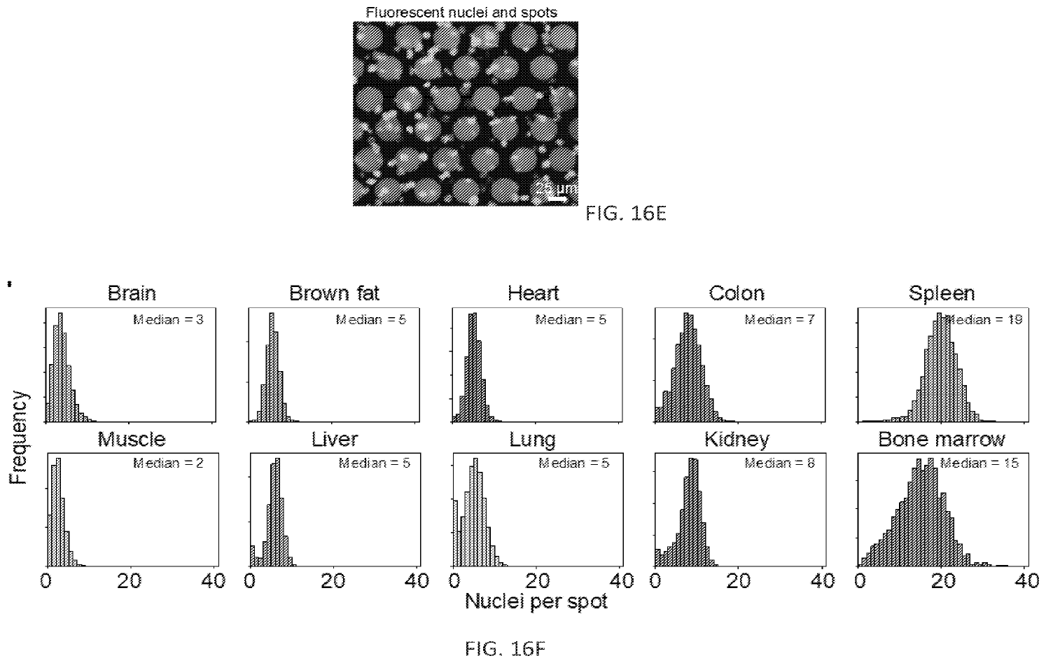

ST profiling was performed on whole mouse sections. Whole mouse sections were transferred onto custom arrays generated as described above. (FIG. 16A-B). Resulting ST data was similar in quality to that observed with mouse kidney (FIG. 15), although the numbers of genes and UMIs detected per spot varied for each organ type at least partially because the numbers of cells present over each spot varies across organs as expected due to variability in cell type sizes (FIG. 16E-F). When focusing on single organs, known anatomical regions were successfully recapitulated by unsupervised clustering (FIG. 16C-D). Results from this experiment provide the first in toto ST dataset of an adult mouse to date. Therefore, the systems and methods described herein combine innovative histological techniques and large format microarrays to provide a tool to study biological processes across whole organisms.

### Example 4

As shown in Example 3, custom ST arrays were generated using on-slide probe assembly to include a spatial barcode for localization, UMIs for quantification, and an oligo (dT) for mRNA capture (FIG. 14). These custom arrays outperform Visium in sensitivity, resolution, and cost. The custom arrays can be further optimized by modifying the sequence composition of the probes on the array to maximize the efficiency of on-slide probe assembly and the sensitivity of downstream ST data.

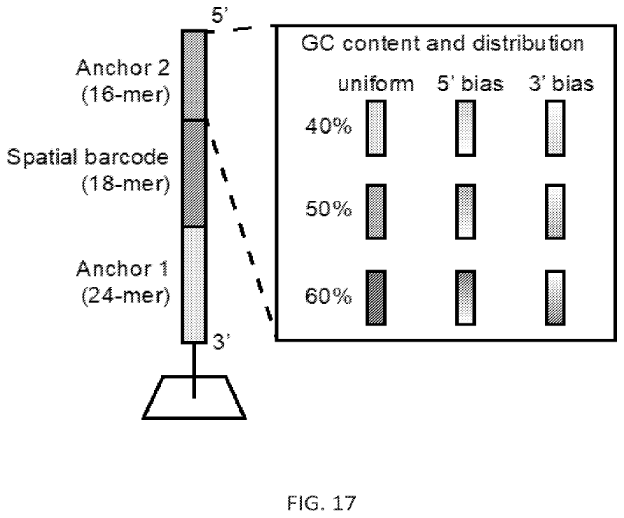

The ST array can be further optimized by modifying the DNA sequence composition of the probes covalently attached to the custom arrays (FIG. 14). The Anchor 1 sequence, tethered to the glass slide by its 3′ end, can be kept constant to match the first 24 bases of the Illumina Read 1 sequencing primer (FIG. 17). The design of Anchor 1 is a modification of published methods for bulk and single cell (sc) RNA-seq^{24,25}, which performed well based on preliminary tests using RNA in solution (e.g., library amplification, library diversity). The length of the Anchor 2 sequence can be kept constant at 16 bases to limit the number of repetitive bases sequenced by the Illumina Read 1 primer between the sequences encoding the Spatial Barcodes and UMIs (FIGS. 14 and 17). In addition, to improve the quality of Illumina sequencing reads by increasing base diversity, PhiX (15%) can be spiked into ST sequencing libraries. While the length of Anchor 2 will remain constant, its sequence can be varied by changing the percentage (40, 50, or 60%) and the distribution of GC bases along the length of the oligo (3′ or 5′ biased or unbiased) (FIG. 17).

Performance of sequence variants can be quantified. For example, to quantify the performance of sequence variants of Anchor 2, purified, total RNA samples can be used, which can be hybridized to custom arrays carrying Anchor 2 variants or the original Anchor 2 sequence as benchmark. For variants of the Anchor 2 sequence, the sequences of the SpatialBarcodes can be designed to avoid self-dimerization issues between Anchor 2, Anchor 1, and Spatial Barcodes. To create Spatial Barcodes, ˜1 million 18-mer sequences (hamming distance ≥3) are generated using the R package DNABarcodes²⁶, which includes various metrics such as self-dimerization, GC %, Tm, and interactions with Anchors. To measure the effects of Anchor 2 sequence variants on the efficiency of on-slide probe assembly, resulting oligo species can be stripped from the array using KOH and analyzed by PAGE as shown above (FIG. 14C). The same conditions for on-slide enzymatic reactions (oligonucleotide 3′ extension and ligation) as in Example 3 can be used to compare the effects of each Anchor 2 design.

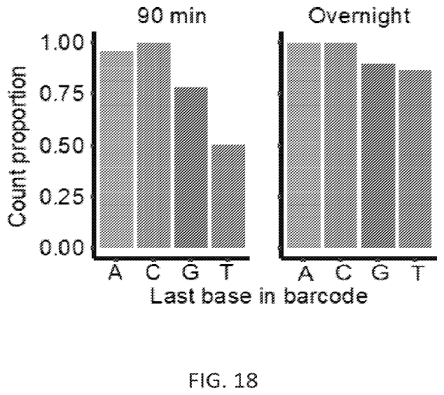

A bias was observed whereby Spatial Barcodes ending in T in their 3′ ends were the least represented in UMI counts per spot (FIG. 18), which is likely due to ligation biases. Optimizing the content and distribution of GC bases along the length of the Anchor 2 sequence may correct those biases by: (i) abrogating residual strand displacement activity (i.e., stripping of annealed Anchor 2 before ligation), if any, of the DNA polymerase (Phusion) which synthesizes the reverse complement of Spatial Barcodes at the 3′-end of Anchor 1 oligos hybridized onto the array; and (ii) maximizing ligation efficiency while minimizing ligation biases towards specific sequences. Optimized Anchor 2 sequences may lead to higher ST output metrics such as the number of UMIs and genes detected by μm².

The on-slide gap-filling reaction steps on the array can also be optimized (FIG. 14B). For example, Phusion DNA polymerase and T4 DNA ligase enzymes can be used for DNA synthesis and ligation, respectively, and the following conditions can be systematically varied: (i) incubation time (1-16 h), (ii) buffer composition (salt concentrations); and (iii) enzyme concentration. Resulting probes can be analyzed by PAGE to determine the proportion of correct and unwanted products (FIG. 14). Longer incubation times helped with decreasing, not abrogating, ligation biases (FIG. 18). Conditions that maximize efficiency while mitigating time and costs can be identified by determining the minimal amount of enzyme needed to prepare an array with optimal yield of the correct capture probes. Lastly, the performance of optimized Anchor 2 sequences and on-slide synthesis procedures can be evaluated by ST profiling of mouse kidneys (as shown in FIG. 15) and sensitivity in UMIs/μm² can be evaluated.

### Example 5

The methods and systems described herein can also be used to generate whole and targeted transcriptome sequencing readouts. The main steps for ST profiling of sections from fresh frozen tissues are H&E staining and imaging, permeabilization followed by cDNA synthesis and amplification, and sequencing library construction. cDNA synthesis and library construction steps can be optimized to generate high quality, whole or targeted transcriptome data. For example, cDNA synthesis and library construction steps can be optimized using a suitable organ, such as mouse kidney sections, as a test system. Permeabilization conditions shown to be suitable for use in a mouse kidney section are shown in FIG. 19, and data (as described above) using a custom microarray on a mouse kidney section has also been generated (FIG. 15).

The conditions for on-slide cDNA synthesis and template switching can be optimized by varying the concentrations of the reverse transcriptase and template switching oligo) and the temperature (42-55° C. using one- or two-step reactions) and the length (30 min to overnight) of the reverse transcription reaction. The conditions for full-length cDNA amplification can also be optimized. In some embodiments, cDNAs are amplified by single-primer PCR amplification using conditions similar to what has been established for bulk and single-cell RNA-seq^{24,25}. The ST methods described herein and RNA-seq methods differ in the length of the partial Illumina Read 1 sequence used in ST (24-mer) vs RNA-seq (33-mer). This length difference is due to the length limit of probes on the custom arrays provided herein (60 bases). The input of cDNA used can vary per single-primer amplification reactions for optimal complexity of resulting next-generation sequencing libraries and downstream sequencing saturation while mitigating the depth of sequencing needed (number of reads per spot). To estimate these metrics from ST data, the median numbers of genes detected per spot as a function of the mean number of reads per spot can be plotted.

Targeted transcriptome analysis can also be performed using the systems and methods described herein. For example, in an exemplary method a gene panel representing low, medium, and high levels of expression in a desired organ can be selected. Biotinylated probes targeting the selected genes can be designed and obtained. Amplified cDNA from the organ can be processed on the ST arrays described herein, hybridized with the biotinylated probes, and target cDNAs can be pulled down (e.g. using streptavidin beads or another biotin-binding agent). Enriched cDNAs can be processed for RNA-seq to quantify the enrichment and expression levels of the gene panel.

In an exemplary method, 500 genes expressed in mouse kidney can be selected to represent low, medium, and high levels of expressions using ST data (FIG. 15) and published RNA-seq data²⁵ on mouse kidney. Biotinylated probes (120-mer) targeting the selected 500 genes at 1× coverage can be designed and obtained, such as by using commercial sources (IDT xGen Hyb Panel Design Tool and kits). Amplified cDNA from kidney sections processed on ST arrays can be hybridized (65° C. overnight) with the biotinylated probes and target cDNAs pulled down, for example using streptavidin beads using a commercial kit (IDT). Enriched cDNAs can be processed for RNA-seq to quantify the enrichment and expression levels of the 500 gene panel.

### Example 6

The methods and systems described herein can be used with formalin-fixed, paraffin-embedded (FFPE) sections. Clinical biospecimens are often preserved as FFPE blocks of tissues or biopsies which highlights the need in biomedical research for spatial profiling methods capable to generate data from FFPE sections. For example, FFPE-compatible ST methods will increase tissue availability and allow for association between ST data and clinical outcomes across patient cohorts. However, FFPE sections typically contain mRNAs in lower quantity and of lesser quality than in fresh frozen counterparts due to RNA fragmentation and long-term storage. To address these challenges, probe hybridization-based methods can be used with the custom ST arrays described herein.

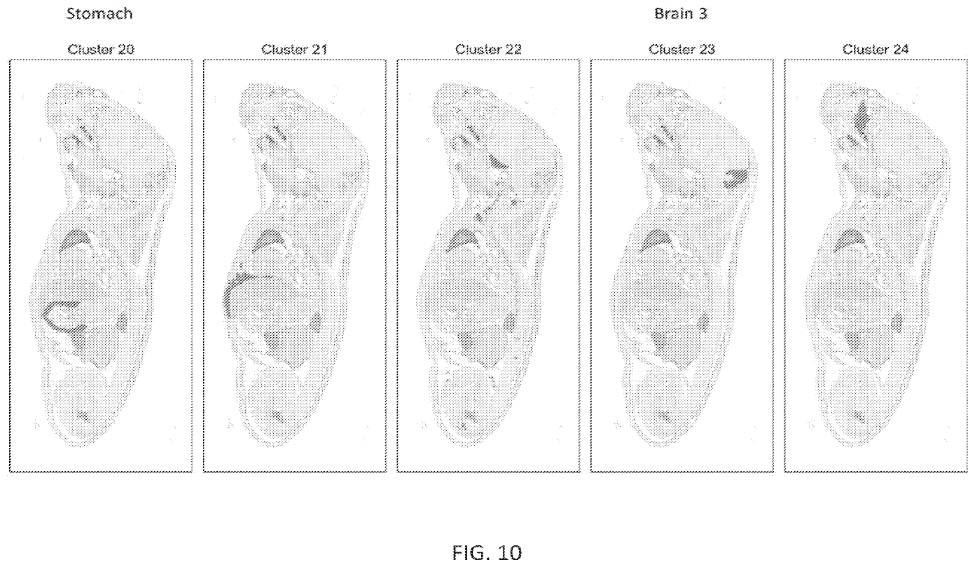

The main steps for ST profiling of sections from FFPE tissues are H&E staining and imaging, oligonucleotide probe hybridization and ligation, RNase treatment and permeabilization, and probe extension followed by next-generation sequencing library construction. In some embodiments, after H&E staining and imaging, FFPE sections can be placed onto custom ST arrays and processed as shown in FIG. 20. Deparaffinization (e.g. using xylene washes) and RNA decrosslinking can be performed (e.g. by incubation at high temperatures with a chelating buffer). Next, probe pairs (e.g. 25-mer each) can be designed and obtained covering desired transcripts. For example, probe pairs can be designed targeting all the transcripts expressed in RNA-seq data for mouse kidney²⁵ using array-based oligo pool synthesis (Agilent). For each pair of probes, one probe carries a poly(dA) sequence for capture on the ST array and another a partial Illumina Read 2 sequence for downstream library construction and sequencing (FIG. 20). Probes can be used for in-tissue hybridization followed by ligation of adjacent probe pairs, RNA degradation, and the capture of ligated probe pairs through poly(dA) on the ST array (FIG. 10). Lastly, the reverse complement of the captured, gene targeting probes can be synthesized at the 3′ end of the ST probes on the array and resulting products will processed for library construction and sequencing (FIG. 20).

### Example 7

The systems and methods described herein can be used to simultaneously measure mRNA and protein levels and to sequence B and T cell receptors (VDJ-seq)^{29,30} from the same histological section. Measuring spatial mRNA and protein levels is valuable to, for example, annotate cell type information using antibodies targeting well-known cell surface markers or study the regulation of gene expression. Sequencing the receptors of lymphocytes in with spatial information will permit studies of immune repertoires, such as host responses to vaccination and infection or infiltrating lymphocytes in tumors.

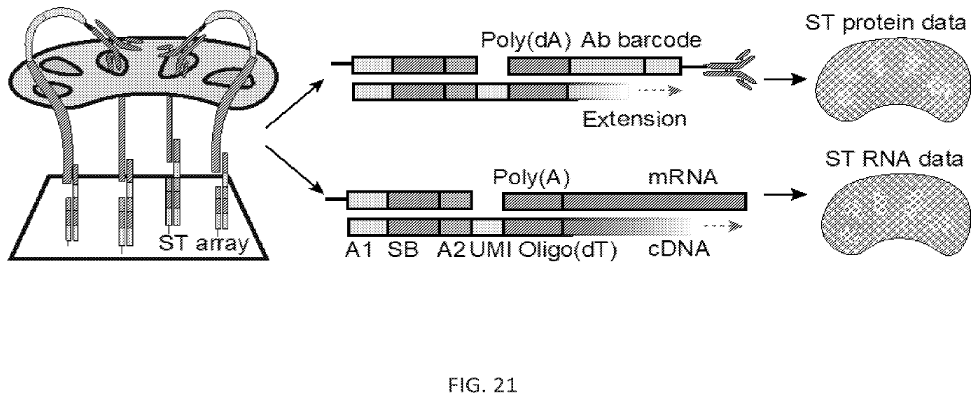

ST methods can be combined with sequencing-based measurements of (i) protein levels using DNA-barcoded antibodies, and/or (ii) immune T and B cell receptor clones. To combine mRNA and protein readouts using sequencing, DNA-barcoded antibodies can be used. Suitable DNA-barcoded antibodies can be obtained from commercial sources, such as BioLegend. Each antibody carries an amplification primer and a barcode sequence followed by a poly(dA) sequence for capture by the oligo (dT) overhang on the array (FIG. 21). Tissues can be obtained and fixed using a suitable fixation buffer to preserve antibody epitopes. For example, tissues can be fixed using 4% paraformaldehyde (PFA) instead of methanol to preserve antibody epitopes. Sections can be stained using standard immunostaining procedures with both fluorescently labeled and DNA-tagged antibodies for the same surface markers (e.g., CD4/8 for T cells, CD19 for B cells). Stained sections can be imaged using a slide scanner compatible with fluorescence prior to permeabilization, cDNA synthesis of tissue mRNAs, and DNA extension of antibody barcodes followed by respective library construction and sequencing.

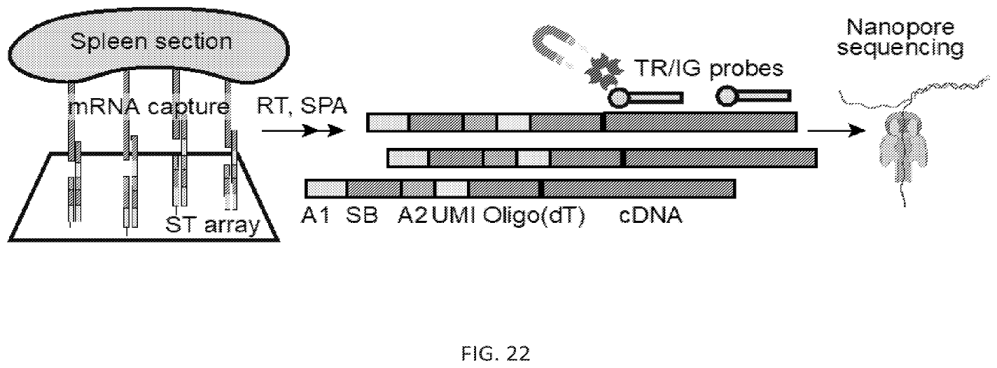

Methods to enrich B and T cell receptor sequences from full-length cDNAs obtained from tissue sections processed ST arrays can be used (FIG. 22). Similar procedures as described above for targeted spatial RNA-seq can be used (Example 5), but using hybridization capture probes (Agilent) targeting B and T cell receptor transcripts. For example, capture probes targeting B and T cell receptor transcripts outside of highly variable CDR3 regions of V genes (i.e., L region, partial V genes, and C genes for all B and T cell receptor chain genes from the IMGT database³²) can be used. Probes can be used to enrich for B and T cell receptor cDNAs from amplified, full-length cDNAs obtained from spleen sections on custom ST arrays. Enriched cDNAs can be processed for long read sequencing (Oxford Nanopore) to determine B and T cell clone sequences (FIG. 22)

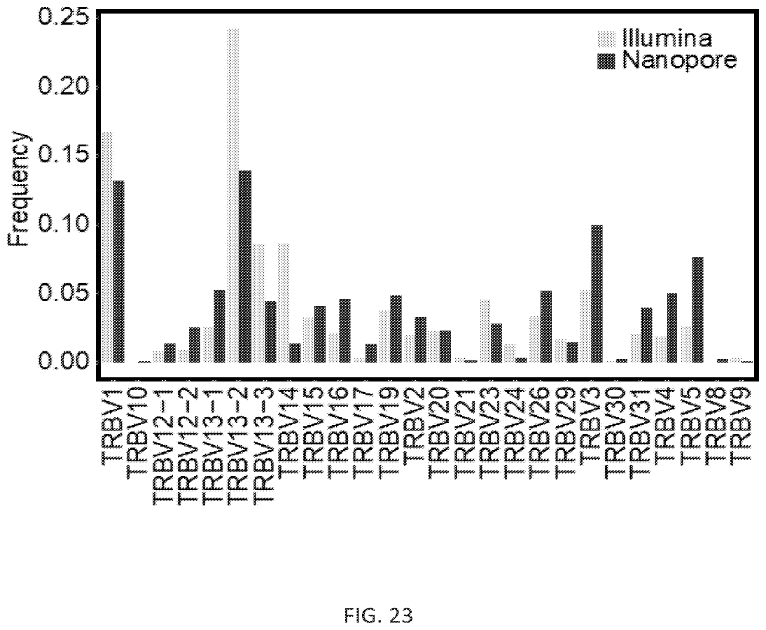

This approach was conducted using 139 probes targeting all mouse TRAV, TRBV, TRAC, and TRBV genes (127 genes for ˜17 kb in combined length). Using this custom probe set on total RNA purified from splenocytes, >70% of the reads matching T cell clonotypes were obtained by long read sequencing (Nanopore 2.0 chemistry for improved base calling accuracy), along with a V gene-usage distribution similar to that obtained by standard short read, TCR-seq methods (FIG. 23).

### Example 8

The spatial analysis of biological tissues and organisms provides understanding of how cell localizations and states are coupled with biological outcomes. For example, cancer progression and remission due to treatment are intimately linked to the cellular and molecular composition and state of tumor lesions. The systems provided herein can be used to evaluate colon samples, including colon samples from patients having colon cancer. The size of the organ is compatible with ST profiling of whole-mount sections. H&E images and ST data from whole-mount sections of human colon specimens containing both normal and cancerous tissues can be obtained. In addition, single-nucleus (sn) RNA-seq data from a tissue section immediately adjacent to the section used for H&E and ST can be obtained.

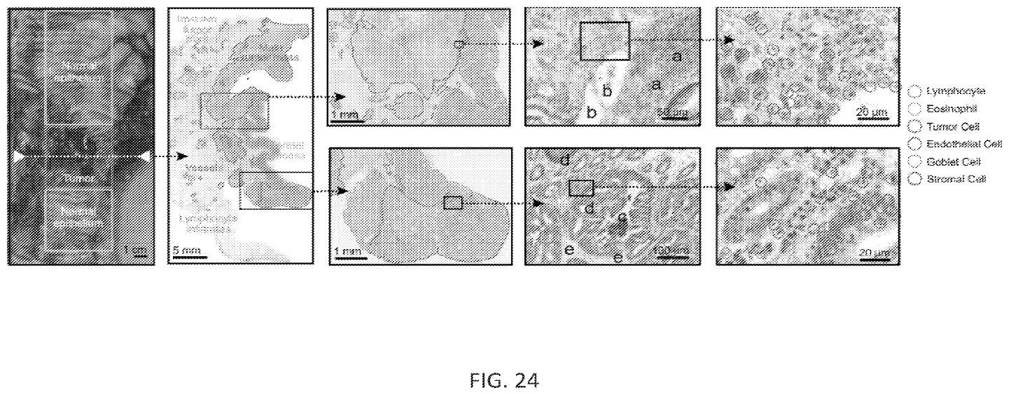

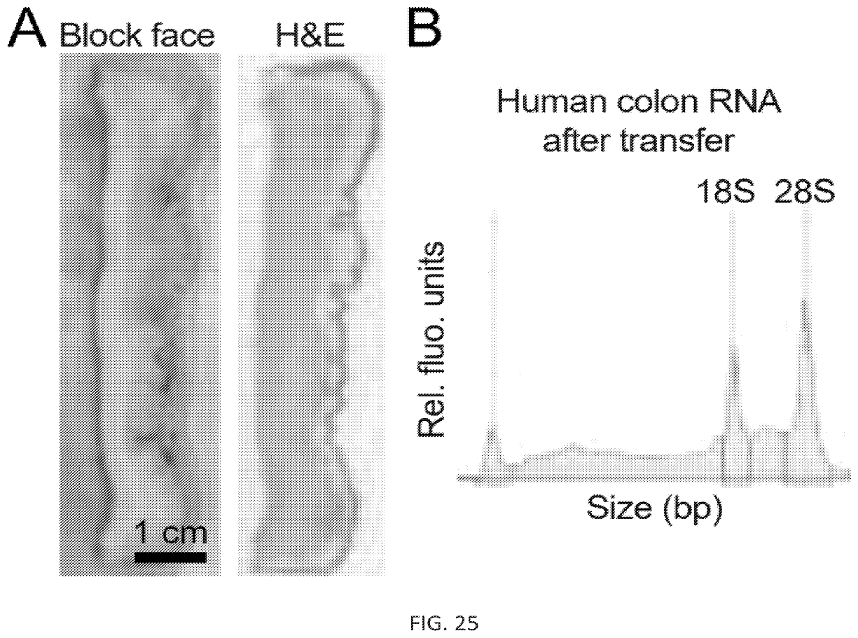

Fresh colon samples can be obtained from patients undergoing surgical resection for colon cancer. A single, full thickness, en bloc ring of tissue containing the cancer lesion and the adjacent normal colon tissue and mesentery can be obtained (FIG. 24). Samples can be fresh frozen and stored until further processing. Samples can be sliced to obtain 10-μm thick, whole-mount sections using a modified cryomacrotome (Leica CM3600 XP) and adhesive film. Sections can be transferred from the adhesive film onto custom large-format arrays, fixed in methanol, stained with H&E, imaged (40 or 100×), and processed for ST profiling. Results demonstrate that high-quality RNA can be obtained from human colon sections (FIG. 25), indicating that these sectioning procedures are compatible with human colon samples. For each colon cancer sample, the following data can be obtained: (1) high-resolution H&E image and ST data (250-500 k ST spots per sample) from the same 10-μm section; and (2) snRNA-seq data (˜50-100 k nuclei across 5-8 punches) from an immediately adjacent 50-μm section. Cell types in normal colon tissues can be identified including, but not limited to, epithelial cells (e.g., goblet cells, colonocytes, Paneth cells, endocrine cells), hematopoietic cells (e.g., lymphocytes, plasma cells, eosinophils, neutrophils, histiocytes, mast cells), stromal cells (e.g., smooth muscle cells, fibroblasts), endothelial cells, neural cells, mesothelial cells, or red blood cells. In colon cancer lesions, we different types of neoplastic epithelial cells which lead to the formation of different subtypes of carcinomas (e.g., adenocarcinoma, squamous cell carcinoma, neuroendocrine carcinoma) can also be identified.

It is understood that the foregoing detailed description is merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

Any patents and publications referenced herein are herein incorporated by reference in their entireties.

Exemplary embodiments in accordance with the disclosure are set forth in the following clauses:
Clause 1: A method of producing a system for spatial detection of nucleic acid in a tissue sample, the method comprising:
a) providing a support comprising an array of surface probes, each surface probe comprising a first anchor sequence, a spatial barcode, and a second anchor sequence;
b) hybridizing a first complementary oligonucleotide to the first anchor sequence of each surface probe;
c) hybridizing a second complementary oligonucleotide to the second anchor sequence of each surface probe, wherein the second oligonucleotide comprises a nucleic acid capture region and a unique molecular identifier; and
d) performing an extension-ligation reaction on the support, wherein the extension-ligation reaction comprises extending the first complementary nucleotide with a sequence complementary to the spatial barcode of the surface probe, and ligating the extended first complementary nucleotide and the second complementary nucleotide together to form a contiguous capture oligonucleotide, the capture oligonucleotide comprising a nucleic acid capture region, a unique molecular identifier, a first anchor sequence, a spatial barcode, and a second anchor sequence.
   Clause 2: The method of clause 1, wherein for each surface probe, the first anchor sequence, the second anchor sequence, and the spatial barcode each comprise 10-30 nucleotides.
   Clause 3: The method of clause 2, wherein for each surface probe the first anchor sequence comprises 20-30 nucleotides, the second anchor sequence comprises 10-20 nucleotides, and the spatial barcode comprises 15-25 nucleotides.
   Clause 4: The method of clause 3, wherein for each surface probe the first anchor sequence comprises 24 nucleotides, the second anchor sequence comprises 16 nucleotides, and the spatial barcode comprises 18 nucleotides.
   Clause 5: The method of any one of clauses 1-4, wherein for each surface probe the second anchor sequence comprises 40% to 60% guanosine and/or cytosine (G/C) bases.
   Clause 6: The method of any one of clauses 1-5, wherein the nucleic acid capture region comprises at least 10 deoxythymidine residues.
   Clause 7: The method of any one of clauses 1-6, wherein the support comprises a glass surface.
   Clause 8: The method of any one of the preceding clauses, wherein performing the extension ligation reaction on the support comprises adding a DNA polymerase and a DNA ligase to the support under conditions such that the reverse complement of the spatial barcode sequence is synthesized and ligated to the first complementary nucleotide and to the second complementary nucleotide, thereby forming the continuous capture probe.
   Clause 8: A system for spatial detection of nucleic acid in a tissue sample, the system comprising a plurality of spots immobilized on a support, wherein:
a) each spot comprises a plurality of capture oligonucleotides,
b) each capture oligonucleotide comprises a nucleic acid capture region, a unique molecular identifier, a first anchor sequence, and a spatial barcode,
c) each capture oligonucleotide in a single spot comprises the same spatial barcode, and
d) the spatial barcode for each distinct spot is unique, wherein the support comprises a working surface area of at least 2 cm².
   Clause 10: The system of clause 9, wherein each capture oligonucleotide further comprises a second anchor sequence.
   Clause 11: The system of clause 9 or clause 10, wherein the support comprises a working surface area of at least 5 cm².
   Clause 12: The system of clause 11, wherein the support comprises a working surface area of at least 10 cm².
   Clause 13: The system of any one of clauses 9-12, wherein the working surface area comprises at least 200 spots/mm².
   Clause 14: The system of clause 13, wherein the working surface area comprises at least 400 spots/mm².
   Clause 15: The system of clause 14, wherein the working surface area comprises at least 800 spots/mm².
   Clause 16: The system of any one of clauses 9-15, wherein the nucleic acid capture region comprises at least 10 deoxythymidine residues.
   Clause 17: The system of any one of clauses 9-16, wherein the nucleic acid is RNA.
   Clause 18: A kit comprising the system of any one of clauses 9-17.
   Clause 19: A method of making the system of any one of clauses 9-17, the method comprising:
a) providing a support comprising an array of surface probes, each surface probe comprising a first anchor sequence, a spatial barcode, and a second anchor sequence;
b) hybridizing a first complementary oligonucleotide to the first anchor sequence of each surface probe;
c) hybridizing a second complementary oligonucleotide to the second anchor sequence of each surface probe, wherein the second oligonucleotide comprises a nucleic acid capture region and a unique molecular identifier;
d) extending the first complementary nucleotide with a sequence complementary to the spatial barcode of the surface probe, such that the first complementary nucleotide and the second complementary nucleotide are ligated form a contiguous capture oligonucleotide, the capture oligonucleotide comprising a nucleic acid capture region, a unique molecular identifier, a first anchor sequence, a spatial barcode, and a second anchor sequence.
   Clause 20: A method for spatial detection of RNA in a tissue sample, the method comprising contacting the system of any one of clauses 9-17 with a tissue sample.
   Clause 21: A method for spatial detection of RNA in a tissue sample, comprising:
a) contacting the system of any one of clauses 9-17 with a tissue sample, such that RNA within the tissue sample to binds to the capture oligonucleotides;
b) reverse-transcribing the bound RNA to generate cDNA; and
c) sequencing the cDNA.
   Clause 22: The method of clause 21, further comprising correlating a spatial barcode for each sequenced cDNA molecule with the location of the spot on the support having a corresponding spatial barcode.
   Clause 23: The method of clause 21 or clause 22, further comprising imaging the tissue and/or staining the tissue before or after sequencing the nucleic acid molecules.
   Clause 24: The method of clause 23, further comprising determining the spatial location of the sequenced cDNA molecules within the tissue sample by correlating the location of the spot on the support with a corresponding location within the tissue sample.
   Clause 25: The method of any one of clauses 20-24, wherein the tissue sample has a surface area of at least 2 cm².
   Clause 26: The method of clause 25, wherein the tissue sample has a surface area of at least 5 cm².
   Clause 27: The method of clause 26, wherein the tissue sample has a surface area of at least 10 cm².
   Clause 28: The method of any one of clauses 20-27, wherein the tissue sample is a fresh frozen tissue sample.
   Clause 29: A method for spatial detection of RNA in a tissue sample, comprising:
a) hybridizing a first probe and a second probe to a target RNA sequence in a tissue sample, wherein the first probe and the second probe each comprise an RNA binding region complementary to the target RNA sequence;
b) ligating the RNA binding region of the first probe to the RNA binding region of the second probe, thereby forming an extended probe that is hybridized to the target RNA sequence in the tissue sample;
c) contacting the tissue sample with the system of any one of clauses 9-17, such that extended probes bind to the capture oligonucleotides;
d) reverse-transcribing the bound extended probes to generate cDNA; and
e) sequencing the cDNA.
   Clause 30: The method of clause 29, wherein the first probe further comprises a capture oligonucleotide binding region complementary to the nucleic acid capture domain of a capture oligonucleotide.
   Clause 31: The method of clause 29 or clause 30, wherein the second probe further comprises a sequencing handle.
   Clause 32: The method of any one of clauses 29-31, further comprising correlating a spatial barcode for each sequenced cDNA molecule with the location of the spot on the support having a corresponding spatial barcode.
   Clause 33: The method of any one of clauses 29-32, further comprising imaging the tissue and/or staining the tissue before or after sequencing the nucleic acid molecules.
   Clause 34: The method of clause 33, further comprising determining the spatial location of the sequenced cDNA molecules within the tissue sample by correlating the location of the spot on the support with a corresponding location within the tissue sample.
   Clause 35: The method of any one of clauses 29-34, wherein the tissue sample has a surface area of at least 2 cm².
   Clause 36: The method of clause 35, wherein the tissue sample has a surface area of at least 5 cm².
   Clause 37: The method of clause 36, wherein the tissue sample has a surface area of at least 10 cm².
   Clause 38: The method of any one of clauses 29-37, wherein the tissue sample is a fresh frozen sample or a formalin-fixed, paraffin-embedded (FFPE) tissue sample.
   Clause 39: The method of clause 38, wherein the tissue sample is an FFPE tissue sample, and wherein the method further comprises deparaffinizing the tissue sample and decrosslinking RNA in the tissue sample prior to hybridizing the first probe and the second probe to the target RNA sequence.
   Clause 40: A method for spatial detection of RNA in a tissue sample, the method comprising:
a) obtaining a tissue sample having a surface area of at least 2 cm², wherein the tissue sample is stabilized on an adhesive film;
b) transferring the tissue sample from the adhesive film to the system of any one of clauses 9-17, such that RNA within the tissue sample to binds to the capture oligonucleotides;
c) reverse-transcribing the bound RNA to generate cDNA; and
d) sequencing the cDNA.
   Clause 41: The method of clause 40, wherein transferring the tissue sample comprises mounting the stabilized tissue sample on the support and dissolving the adhesive film in hexane, thereby transferring the tissue sample from the adhesive film to the support.
   Clause 42: The method of clause 40 or clause 41, further comprising correlating a spatial barcode for each sequenced cDNA molecule with the location of the spot on the support having a corresponding spatial barcode.
   Clause 43: The method of any one of clauses 40-42, further comprising imaging the tissue and/or staining the tissue after transferring the tissue sample from the adhesive film to the support.
   Clause 44: The method of clause 43, wherein the tissue is imaged and/or stained before or after sequencing the cDNA.
   Clause 45: The method of clause 43 or clause 44 further comprising determining the spatial location of the sequenced cDNA molecules within the tissue sample by correlating the location of the spot on the support with a corresponding location within the tissue sample.
   Clause 46: The method of any one of clauses 40-45, wherein the tissue sample has a surface area of at least 2 cm².
   Clause 47: The method of clause 46, wherein the tissue sample has a surface area of at least 5 cm².
   Clause 48: The method of clause 47, wherein the tissue sample has a surface area of at least 10 cm².
   Clause 49: The method of any one of clauses 40-48, wherein the tissue sample is a fresh frozen tissue sample.
   Clause 50: A method for spatial detection of RNA in a tissue sample, comprising:
a) obtaining a tissue sample having a surface area of at least 2 cm², wherein the tissue sample is stabilized on an adhesive film;
b) transferring the tissue sample from the adhesive film to the system of any one of clauses 9-17, wherein transferring the tissue sample comprises mounting the stabilized tissue sample on the support and dissolving the adhesive film in hexane, thereby transferring the tissue sample from the adhesive film to the support;
c) hybridizing a first probe and a second probe to a target RNA sequence in the tissue sample, wherein the first probe and the second probe each comprise an RNA binding region complementary to the target RNA sequence;
d) ligating the RNA binding region of the first probe to the RNA binding region of the second probe, thereby forming an extended probe that is hybridized to the target RNA sequence in the tissue sample,
e) permitting extended probes to bind to capture oligonucleotides on the on the support,
f) reverse-transcribing the bound extended probes to generate cDNA; and
g) sequencing the cDNA.
   Clause 51: The method of clause 50, wherein the first probe further comprises a capture oligonucleotide binding region complementary to the nucleic acid capture domain of a capture oligonucleotide.
   Clause 52: The method of clause 50 or clause 51, wherein the second probe further comprises a sequencing handle.
   Clause 53: The method of any one of clauses 50-52, further comprising correlating a spatial barcode for each sequenced cDNA molecule with the location of the spot on the support having a corresponding spatial barcode.
   Clause 54: The method of clause 53, further comprising imaging and/or staining the tissue after transferring the tissue sample from the adhesive film to the support.
   Clause 55: The method of clause 54, wherein the tissue is imaged and/or stained before or after sequencing the cDNA.
   Clause 56: The method of clause 54 or clause 55, further comprising determining the spatial location of the sequenced cDNA molecules within the tissue sample by correlating the location of the spot on the support with a corresponding location within the tissue sample.
   Clause 57: The method of any one of clauses 50-56, wherein the tissue sample has a surface area of at least 2 cm².
   Clause 58: The method of clause 57, wherein the tissue sample has a surface area of at least 5 cm².
   Clause 59: The method of clause 58, wherein the tissue sample has a surface area of at least 10 cm².
   Clause 60: The method of any one of clauses 50-59, wherein the tissue sample is a fresh frozen sample or a formalin-fixed, paraffin-embedded (FFPE) tissue sample.
   Clause 61: The method of clause 60, wherein the tissue sample is an FFPE tissue sample, and wherein the method further comprises deparaffinizing the tissue sample and decrosslinking RNA in the tissue sample prior to hybridizing the first probe and the second probe to the target RNA sequence.

### REFERENCES

(1) Regev, A.; Teichmann, S. A.; Lander, E. S.; Amit, I.; Benoist, C.; Birney, E.; Bodenmiller, B.; Campbell, P.; Carninci, P.; Clatworthy, M., et al. The Human Cell Atlas. Elife 2017, 6.
(2) HuBMAP Consortium. The human body at cellular resolution: the NIH Human Biomolecular Atlas Program. Nature 2019, 574 (7777), 187-192.
(3) Lein, E.; Borm, L. E.; Linnarsson, S. The promise of spatial transcriptomics for neuroscience in the era of molecular cell typing. Science 2017, 358 (6359), 64-69.
(4) Ståhl, P. L.; Salmen, F.; Vickovic, S.; Lundmark, A.; Navarro, J. F.; Magnusson, J.; Giacomello, S.; Asp, M.; Westholm, J. O.; Huss, M.; et al. Visualization and analysis of gene expression in tissue sections by spatial transcriptomics. Science 2016, 353 (6294), 78-82.
(5) Rodriques, S. G.; Stickels, R. R.; Goeva, A.; Martin, C. A.; Murray, E.; Vanderburg, C. R.; Welch, J.; Chen, L. M.; Chen, F.; Macosko, E. Z. Slide-seq: A scalable technology for measuring genome-wide expression at high spatial resolution. Science 2019, 363 (6434), 1463-1467.
(6) Vickovic, S.; Eraslan, G.; Salmen, F.; Klughammer, J.; Stenbeck, L.; Schapiro, D.; Äijö, T.; Bonneau, R.; Bergenstråhle, L.; Navarro, J. F.; et al. High-definition spatial transcriptomics for in situ tissue profiling. Nat Methods 2019, 16 (10), 987-990.
(7) Liu, Y.; Yang, M.; Deng, Y.; Su, G.; Enninful, A.; Guo, C. C.; Tebaldi, T.; Zhang, D.; Kim, D.; Bai, Z.; et al. High-Spatial-Resolution Multi-Omics Sequencing via Deterministic Barcoding in Tissue. Cell 2020, 183 (6), 1665-1681.
(8) Stickels, R. R.; Murray, E.; Kumar, P.; Li, J.; Marshall, J. L.; Di Bella, D. J.; Arlotta, P.; Macosko, E. Z.; Chen, F. Highly sensitive spatial transcriptomics at near-cellular resolution with Slide-seq V2. Nat Biotechnol 2021, 39 (3), 313-319.
(9) Chen, A.; Liao, S.; Cheng, M.; Ma, K.; Wu, L.; Lai, Y.; Qiu, X.; Yang, J.; Xu, J.; Hao, S.; et al. Spatiotemporal transcriptomic atlas of mouse organogenesis using DNA nanoball-patterned arrays. Cell 2022, 185 (10), 1777-1792 e1721.
(10) Salmen, F.; Stahl, P. L.; Mollbrink, A.; Navarro, J. F.; Vickovic, S.; Frisen, J.; Lundeberg, J. Barcoded solid-phase RNA capture for Spatial Transcriptomics profiling in mammalian tissue sections. Nat Protoc 2018, 13 (11), 2501-2534.
(11) Moses, L.; Pachter, L. Museum of spatial transcriptomics. Nat Methods 2022, 19 (5), 534-546.
(12) Shaukat, A.; Levin, T. R. Current and future colorectal cancer screening strategies. Nat Rev Gastroenterol Hepatol 2022, 19 (8), 521-531.
(13) Ullberg, S. Studies on the distribution and fate of S35-labelled benzylpenicillin in the body. Acta Radiol Suppl 1954, 118, 1-110.
(14) Kawamoto, T. Light microscopic autoradiography for study of early changes in the distribution of water-soluble materials. J Histochem Cytochem 1990, 38 (12), 1805-1814.
(15) Kawamoto, T. Use of a new adhesive film for the preparation of multi-purpose fresh-frozen sections from hard tissues, whole-animals, insects and plants. Arch Histol Cytol 2003, 66 (2), 123-143.
(16) Kawamoto, T.; Kawamoto, K. Preparation of thin frozen sections from nonfixed and undecalcified hard tissues using Kawamot's film method (2012). Methods Mol Biol 2014, 1130, 149-164.
(17) Kawamoto, T.; Kawamoto, K. Preparation of Thin Frozen Sections from Nonfixed and Undecalcified Hard Tissues Using Kawamoto's Film Method (2020). Methods Mol Biol 2021, 2230, 259-281.
(18) Islam, S.; Zeisel, A.; Joost, S.; La Manno, G.; Zajac, P.; Kasper, M.; Lönnerberg, P.; Linnarsson, S. Quantitative single-cell RNA-seq with unique molecular identifiers. Nat Methods 2014, 11 (2), 163-166.
(19) Zhu, Y. Y.; Machleder, E. M.; Chenchik, A.; Li, R.; Siebert, P. D. Reverse transcriptase template switching: a SMART approach for full-length cDNA library construction. Biotechniques 2001, 30 (4), 892-897.
(20) Kaminow, B.; Yunusov, D.; Dobin, A. STARsolo: accurate, fast and versatile mapping/quantification of single-cell and single-nucleus RNA-seq data. bioRxiv 2021, 2021.05.05.442755
(21) Wolf, F. A.; Angerer, P.; Theis, F. J. SCANPY: large-scale single-cell gene expression data analysis. Genome Biol 2018, 19 (1), 15.
(22) van der Walt, S.; Schonberger, J. L.; Nunez-Iglesias, J.; Boulogne, F.; Warner, J. D.; Yager, N.; Gouillart, E.; Yu, T.; scikit-image, c. scikit-image: image processing in Python. PeerJ 2014, 2, e453.
(23) Waskom, M. L. seaborn: statistical data visualization. Journal of Open Source Software 2021, 6 (60), 3021.
(24) Kadoki, M.; Patil, A.; Thaiss, C. C.; Brooks, D. J.; Pandey, S.; Deep, D.; Alvarez, D.; von Andrian, U. H.; Wagers, A. J.; Nakai, K.; et al. Organism-Level Analysis of Vaccination Reveals Networks of Protection across Tissues. Cell 2017, 171 (2), 398-413.
(25) Pandey, S.; Takahama, M.; Gruenbaum, A.; Zewde, M.; Cheronis, K.; Chevrier, N. A whole-tissue RNA-seq toolkit for organism-wide studies of gene expression with PME-seq. Nat Protoc 2020, 15 (4), 1459-1483.
(26) Buschmann, T. DNABarcodes: an R package for the systematic construction of DNA sample tags. Bioinformatics 2017, 33 (6), 920-922.
(27) Mathieson, W.; Thomas, G. A. Why Formalin-fixed, Paraffin-embedded Biospecimens Must Be Used in Genomic Medicine: An Evidence-based Review and Conclusion. J Histochem Cytochem 2020, 68 (8), 543-552.
(28) Su, G.; Qin, X.; Enninful, A.; Bai, Z.; Deng, Y.; Liu, Y.; Fan, R. Spatial multi-omics sequencing for fixed tissue via DBiT-seq. STAR Protoc 2021, 2 (2), 100532.
(29) Bradley, P.; Thomas, P. G. Using T Cell Receptor Repertoires to Understand the Principles of Adaptive Immune Recognition. Annu Rev Immunol 2019, 37, 547-570.
(30) Magadan, S. Adaptive immune receptor repertoires, an overview of this exciting field. Immunol Lett 2020, 221, 49-55.
(31) Stoeckius, M.; Hafemeister, C.; Stephenson, W.; Houck-Loomis, B.; Chattopadhyay, P. K.; Swerdlow, H.; Satija, R.; Smibert, P. Simultaneous epitope and transcriptome measurement in single cells. Nat Methods 2017, 14 (9), 865-868.
(32) Lefranc, M. P. Immunoglobulin and T Cell Receptor Genes: IMGT((R)) and the Birth and Rise of Immunoinformatics. Front Immunol 2014, 5, 22.
(33) Vickovic, S.; Lotstedt, B.; Klughammer, J.; Mages, S.; Segerstolpe, A.; Rozenblatt-Rosen, O.; Regev, A. SM-Omics is an automated platform for high-throughput spatial multi-omics. Nat Commun 2022, 13 (1), 795.
(34) Kleshchevnikov, V.; Shmatko, A.; Dann, E.; Aivazidis, A.; King, H. W.; Li, T.; Elmentaite, R.; Lomakin, A.; Kedlian, V.; Gayoso, A.; et al. Cell2location maps fine-grained cell types in spatial transcriptomics. Nat Biotechnol 2022, 40 (5), 661-671.
(35) Cable, D. M.; Murray, E.; Zou, L. S.; Goeva, A.; Macosko, E. Z.; Chen, F.; Irizarry, R. A. Robust decomposition of cell type mixtures in spatial transcriptomics. Nat Biotechnol 2022, 40 (4), 517-526.
(36) Shmatko, A.; Ghaffari Laleh, N.; Gerstung, M.; Kather, J. N. Artificial intelligence in histopathology: enhancing cancer research and clinical oncology. Nat Cancer 2022, 3 (9), 1026-1038.
(37) Liu, Y.; Carbonetto, P.; Takahama, M.; Gruenbaum, A.; Xie, D.; Chevrier, N.; Stephens, M. A flexible model for correlated count data, with application to analysis of gene expression differences in multi-condition experiments. arXiv 2022, 2210.00697.
(38) Miller, B. F.; Huang, F.; Atta, L.; Sahoo, A.; Fan, J. Reference-free cell type deconvolution of multi-cellular pixel-resolution spatially resolved transcriptomics data. Nat Commun 2022, 13 (1), 2339.
(39) Dey, K. K.; Hsiao, C. J.; Stephens, M. Visualizing the structure of RNA-seq expression data using grade of membership models. PLoS Genet 2017, 13 (3), e1006599.
(40) Al-Asadi, H.; Dey, K. K.; Novembre, J.; Stephens, M. Inference and visualization of DNA damage patterns using a grade of membership model. Bioinformatics 2019, 35 (8), 1292-1298.

We claim:

1. A method of producing a system for spatial detection of nucleic acid in a tissue sample, the method comprising:
a) providing a support comprising an array of surface probes, each surface probe comprising a first anchor sequence, a spatial barcode, and a second anchor sequence;
b) hybridizing a first complementary oligonucleotide to the first anchor sequence of each surface probe;
c) hybridizing a second complementary oligonucleotide to the second anchor sequence of each surface probe, wherein the second oligonucleotide comprises a nucleic acid capture region and a unique molecular identifier; and
d) performing an extension-ligation reaction on the support, wherein the extension-ligation reaction comprises extending the first complementary nucleotide with a sequence complementary to the spatial barcode of the surface probe, and ligating the extended first complementary nucleotide and the second complementary nucleotide together to form a contiguous capture oligonucleotide, the capture oligonucleotide comprising a nucleic acid capture region, a unique molecular identifier, a first anchor sequence, a spatial barcode, and a second anchor sequence.

2. The method of claim 1, wherein for each surface probe, the first anchor sequence, the second anchor sequence, and the spatial barcode each comprise 10-30 nucleotides.

3. The method of claim 2, wherein for each surface probe the first anchor sequence comprises 20-30 nucleotides, the second anchor sequence comprises 10-20 nucleotides, and the spatial barcode comprises 15-25 nucleotides.

4. The method of claim 3, wherein for each surface probe the first anchor sequence comprises 24 nucleotides, the second anchor sequence comprises 16 nucleotides, and the spatial barcode comprises 18 nucleotides.

5. The method of any one of claims 1-4, wherein for each surface probe the second anchor sequence comprises 40% to 60% guanosine and/or cytosine (G/C) bases.

6. The method of any one of claims 1-5, wherein the nucleic acid capture region comprises at least 10 deoxythymidine residues.

7. The method of any one of claims 1-6, wherein the support comprises a glass surface.

8. The method of any one of the preceding claims, wherein performing the extension ligation reaction on the support comprises adding a DNA polymerase and a DNA ligase to the support under conditions such that the reverse complement of the spatial barcode sequence is synthesized and ligated to the first complementary nucleotide and to the second complementary nucleotide, thereby forming the continuous capture probe.

9. A system for spatial detection of nucleic acid in a tissue sample, the system comprising a plurality of spots immobilized on a support, wherein:
a) each spot comprises a plurality of capture oligonucleotides,
b) each capture oligonucleotide comprises a nucleic acid capture region, a unique molecular identifier, a first anchor sequence, and a spatial barcode,
c) each capture oligonucleotide in a single spot comprises the same spatial barcode, and
d) the spatial barcode for each distinct spot is unique,
wherein the support comprises a working surface area of at least 2 cm².

10. The system of claim 9, wherein each capture oligonucleotide further comprises a second anchor sequence.

11. The system of claim 9 or claim 10, wherein the support comprises a working surface area of at least 5 cm².

12. The system of claim 11, wherein the support comprises a working surface area of at least 10 cm².

13. The system of any one of claims 9-12, wherein the working surface area comprises at least 200 spots/mm².

14. The system of claim 13, wherein the working surface area comprises at least 400 spots/mm².

15. The system of claim 14, wherein the working surface area comprises at least 800 spots/mm².

16. The system of any one of claims 9-15, wherein the nucleic acid capture region comprises at least 10 deoxythymidine residues.

17. The system of any one of claims 9-16, wherein the nucleic acid is RNA.

18. A kit comprising the system of any one of claims 9-17.

19. A method of making the system of any one of claims 9-17, the method comprising:
a) providing a support comprising an array of surface probes, each surface probe comprising a first anchor sequence, a spatial barcode, and a second anchor sequence;
b) hybridizing a first complementary oligonucleotide to the first anchor sequence of each surface probe;
c) hybridizing a second complementary oligonucleotide to the second anchor sequence of each surface probe, wherein the second oligonucleotide comprises a nucleic acid capture region and a unique molecular identifier;
d) extending the first complementary nucleotide with a sequence complementary to the spatial barcode of the surface probe, such that the first complementary nucleotide and the second complementary nucleotide are ligated form a contiguous capture oligonucleotide, the capture oligonucleotide comprising a nucleic acid capture region, a unique molecular identifier, a first anchor sequence, a spatial barcode, and a second anchor sequence.

20. A method for spatial detection of RNA in a tissue sample, the method comprising contacting the system of any one of claims 9-17 with a tissue sample.

21. A method for spatial detection of RNA in a tissue sample, comprising:
a) contacting the system of any one of claims 9-17 with a tissue sample, such that RNA within the tissue sample to binds to the capture oligonucleotides;
b) reverse-transcribing the bound RNA to generate cDNA; and
c) sequencing the cDNA.

22. The method of claim 21, further comprising correlating a spatial barcode for each sequenced cDNA molecule with the location of the spot on the support having a corresponding spatial barcode.

23. The method of claim 21 or claim 22, further comprising imaging the tissue and/or staining the tissue before or after sequencing the nucleic acid molecules.

24. The method of claim 23, further comprising determining the spatial location of the sequenced cDNA molecules within the tissue sample by correlating the location of the spot on the support with a corresponding location within the tissue sample.

25. The method of any one of claims 20-24, wherein the tissue sample has a surface area of at least 2 cm².

26. The method of claim 25, wherein the tissue sample has a surface area of at least 5 cm².

27. The method of claim 26, wherein the tissue sample has a surface area of at least 10 cm².

28. The method of any one of claims 20-27, wherein the tissue sample is a fresh frozen tissue sample.

29. A method for spatial detection of RNA in a tissue sample, comprising:
a) hybridizing a first probe and a second probe to a target RNA sequence in a tissue sample, wherein the first probe and the second probe each comprise an RNA binding region complementary to the target RNA sequence;
b) ligating the RNA binding region of the first probe to the RNA binding region of the second probe, thereby forming an extended probe that is hybridized to the target RNA sequence in the tissue sample;
c) contacting the tissue sample with the system of any one of claims 9-17, such that extended probes bind to the capture oligonucleotides;
d) reverse-transcribing the bound extended probes to generate cDNA; and
e) sequencing the cDNA.

30. The method of claim 29, wherein the first probe further comprises a capture oligonucleotide binding region complementary to the nucleic acid capture domain of a capture oligonucleotide.

31. The method of claim 29 or claim 30, wherein the second probe further comprises a sequencing handle.

32. The method of any one of claims 29-31, further comprising correlating a spatial barcode for each sequenced cDNA molecule with the location of the spot on the support having a corresponding spatial barcode.

33. The method of any one of claims 29-32, further comprising imaging the tissue and/or staining the tissue before or after sequencing the nucleic acid molecules.

34. The method of claim 33, further comprising determining the spatial location of the sequenced cDNA molecules within the tissue sample by correlating the location of the spot on the support with a corresponding location within the tissue sample.

35. The method of any one of claims 29-34, wherein the tissue sample has a surface area of at least 2 cm².

36. The method of claim 35, wherein the tissue sample has a surface area of at least 5 cm².

37. The method of claim 36, wherein the tissue sample has a surface area of at least 10 cm².

38. The method of any one of claims 29-37, wherein the tissue sample is a fresh frozen sample or a formalin-fixed, paraffin-embedded (FFPE) tissue sample.

39. The method of claim 38, wherein the tissue sample is an FFPE tissue sample, and wherein the method further comprises deparaffinizing the tissue sample and decrosslinking RNA in the tissue sample prior to hybridizing the first probe and the second probe to the target RNA sequence.
